(19) United States
(12) Patent Application Publication
(10) Pub. No.: US 2003/0149096 A1
(43) Pub. Date: Aug. 7, 2003

(54) Cancer chemopreventative compounds and compositions and methods of treating cancers

(76) Inventors: John M. Pezzuto, River Forest, IL (US); James D. McChesney, Boulder, CO (US); Muriel Anne Cuendet, Chicago, IL (US); Lawrence Helson, Quaker Town, PA (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/200,370

(22) Filed: Jul. 22, 2002

Related U.S. Application Data
(63) Continuation-in-part of application No. 10/066,809, filed on Feb. 4, 2002.
(60) Provisional application No. 60/266,543, filed on Feb. 5, 2001.

(51) Int. Cl. A61K 31/366

(52) U.S. Cl. 514/453

(57) ABSTRACT

A composition and method of treating multiple myeloma are disclosed. The composition and method utilize brusatol, bruceantin, glaucarubolone, and derivatives thereof as active multiple myeloma treating agents in mammals, including humans.

### CROSS REFERENCE TO RELATED APPLICATIONS

This application is a continuation-in-part of copending application Serial No. 10/066,809, filed Feb. 4, 2002, which claims the benefit of U.S. provisional patent application Serial No. 60/266,543, filed Feb. 5, 2001.

### STATEMENT OF GOVERNMENTAL INTEREST

This work was made with government support under by Grant No. PO1 CA48112 awarded by the NCI, National Institutes of Health, Bethesda, Md.

### FIELD OF THE INVENTION

The present invention relates to cancer chemopreventive therapeutic compositions and methods. More particularly, the present invention relates to cancer chemoprevention and cancer therapy in mammals, including humans, utilizing brusatol, glaucarubolone, and derivatives thereof as cancer chemopreventive and cancer therapeutic agents, especially in the treatment of multiple myeloma.

### BACKGROUND OF THE INVENTION

Cancer claims millions of lives each year and is the largest single cause of death in both men and women. Extrinsic factors, including personal lifestyles, play a major role in the development of most human malignancies. Cigarette smoking, consumption of alcohol, exposure to synthetic and naturally occurring carcinogens, radiation, drugs, infectious agents, and reproductive and behavioral practices are widely recognized as important contributors to the etiology of cancer.

Chemoprevention, i.e., the prevention of cancer by administration of chemical agents that reduce the risk of carcinogenesis is one of the most direct ways to reduce cancer-related morbidity and mortality. See, M. B. Sporn, Fed. Proc., 38, 2528 (1979). However, chemoprevention requires the identification of carcinogens and chemopreventatives, even though interactions between the factors that modulate cancer risk are complex. Whereas extensive efforts have been made to identify carcinogens and mutagens, the identification of chemo-preventative agents has received less attention.

Cancer chemopreventive agents include nonsteroidal antiinflammatory drugs (NSAIDs), such as indomethacin, aspirin, piroxicam, and sulindac, all of which inhibit cyclooxygenase. There is a need in the art, however, for the identification of additional specific compounds that have a cancer chemopreventative effect on mammals. Such cancer chemopreventative compounds then can be used in drug compositions to reduce the risk of, or to treat, a cancer.

There also is a need for improved cancer therapeutic agents. Agents used for the treatment of existing cancers typically mediate substantial adverse side effects, whereas cancer chemopreventive agents generally are less toxic. However, if an existing cancer can be treated with a chemopreventative agent, such an agent should also be categorized as a cancer chemotherapeutic agent. Mechanistic-based agents, such as those described herein, fall into this category.

Hematologic malignancies include a diverse number of cancers. These malignancies have been the focus of intense investigation with respect to providing improved chemopreventative and chemotherapeutic agents. The following are various types of hematologic malignancies requiring improved chemopreventative and chemotherapeutic agents:

Acute myeloid leukemia. Acute myeloid leukemia (AML) is the cause of approximately 1.2% of all cancer deaths in the U.S., with an annual incidence rate of 2.2 per 100,000 and approximately 9,200 new cases per year representing approximately 90% of all acute leukemias in adults. The incidence rises with age, genetic predisposition, drug and environmental exposures, and occupational factors may have a role in its genesis.

Standard therapy of AML includes remission induction with regimens consisting of ara-C and an anthracycline followed by consolidation with similar regimens. The most important predictor of outcome after relapse is the length of the initial complete remission (CR). For patients whose initial CR lasted greater than 2 years, repeating the initial regimen can result in a 50-60% CR rate, whereas those whose CR was less than 1 year can expect only a 10-20% CR rate with such an approach. Prognosis of patients with refractory disease (i.e., no CR after two courses of induction) is quantitatively similar to those with the short first CR with the best results in those receiving an allogeneic transplant. The general consensus is that those patients with primary refractory disease or a short first CR should receive an allogeneic transplant if a suitable donor is available and without waiting for a second CR. If an allogeneic transplant cannot be performed, these patients should be offered investigational therapies. In patients who relapse after an allogeneic transplant, the prognosis also depends on the length of CR. Donor lymphocyte infusions can produce remissions in some patients. Another therapy for patients who have a CR duration greater than 1 year is a second allogeneic transplant. Otherwise, and in patients with CR less than a year, investigational therapies should be offered.

Acute lymphoblastic leukemia (ALL). The age-adjusted overall incidence of ALL in the U.S., is 2.3/100,000. After a first peak in children younger than (5.3/100,000), the incidence decreases until a second minor peak at age 80-84 (2.3/-100,000). Treatment of ALL involves remission induction, intensification, maintenance, and CNS prophylaxis. Therapy of patients with refractory disease and those who relapse has involved a number of different regimens usually containing high dose ara-C or methotrexate combined with other agents, such as idarubicin, fludarabine, and asparaginase, with varying response rates and durations. The most significant predictor for response to therapy following relapse in ALL is the duration of first CR with patients having a longer than 18 months duration having higher response and longer remission duration. The role of allogeneic transplant in ALL is not fully established. However, one approach is to for patients with low-risk disease to receive transplant only at the time of relapse and second remission and high-risk patients (e.g., those with Ph+disease) to receive transplant in first remission. Use of investigational agents in more advanced situations than those described above should be a priority.

Blast phase Chronic myeloid leukemia (CML). The treatment of blast crisis in CML remains unsatisfactory. However, the distinction between lymphoid and myeloid blast crisis has important therapeutic implications. In two-thirds of the patients, the transformed cells have myeloid or undifferentiated markers and should be treated with cytarabine-based regimens for acute myeloid leukemia, preferably on a clinical trial. These patients have low response rates and short survival. The remainder of patients with lymphoid markers have a better response and outcome when treated with regimens for acute lymphoblastic leukemia. These patients are best treated on clinical trials with consideration for consolidation with an allogeneic transplant after achieving a remission. Novel agents such as decitabine have been investigated for the treatment of patients with accelerated and blastic disease with myeloid markers and significant responses have been reported. Results of early studies of the tyrosine kinase inhibitor STI571 in blast phase CML have recently been reported. Unfortunately, most responses were transient lasting a median of 3 months only, and few responses in myeloid patients lasting more than a year. Therefore, the search for other agents with significant activity against this disease, which alone or in combination with STI571, would improve the response rate and duration, continues.

Burkitt's and Burkitt-like leukemia/-lymphoma. In the working formulation classification of lymphomas, aggressive or “high grade” lymphomas included diffuse small noncleaved lymphomas (DSNCL), as well lymphoblastic and immunoblastic lymphomas. The entity DSNCL has been subdivided in the REAL classification into Burkitt's lymphoma and high grade B-cell lymphoma, Burkitt-like. This subdivision is based largely upon the degree of cellular pleomorphism, and it is not clear that such a separation has prognostic significance. Three variants of Burkitt's disease have been described: the endemic (African), sporadic (American), and AIDS-related. The morphology of Burkitt's lymphoma cells is very similar to the L-3 subtype of ALL, with the cells being mature B-cells with the expression of surface immunoglobulin. The disease is associated with chromosomal translocations involving the c-Myc oncogene and immunoglobulin genes. One of three alternative forms of the immunoglobulin/myc translocation—8:14 (myc/IgH), 2:8 (k/myc), and 8:22 (myc/l)—are regularly present in all Burkitt lymphomas. The subordination of c-myc to one of the continuously active immunoglobulin regions interferes with the normal regulation of the gene and its over-expression. As a result, the cells are prevented from leaving the cycling compartment. This translocation, therefore, is considered the main rate-limiting event in the development of Burkitt's lymphoma. DSNCL of the non-Burkitt's-type have a histologic appearance and cytogenetic findings intermediate between and overlapping Burkitt's lymphoma and large cell lymphomas of B-cell origin. Lymphoblastic lymphomas (LBL) mostly have an immature T-cell phenotype, although precursor B-cell phenotypes have been described. Cytogenetically T-cell LBL is similar to T-cell ALL. The primary treatment modality for DSNCLs is chemotherapy, regardless of the site of disease. The current practice is to use short duration, intensive combination chemotherapy regimens and with the most effective regimens in use 90%-100% of patients with limited disease, and 80%-90% of patients with advanced disease can be cured. Bone marrow transplantation is considered only as a salvage treatment option for relapsing patients, or in patients with unresponsive disease. With excellent results of modern chemotherapy, consolidation with high dose therapy, even in patients with extensive disease, is generally felt to be unnecessary. It is not clear that autologous transplantation is the ideal therapy for relapsed patients with only a minority obtaining a benefit from this procedure. Allogeneic transplant has a cure potential but is associated with significant morbidity and mortality. Use of investigational agents in patients with relapsed/-refractory disease who are unable or unwilling to undergo transplantation is warranted.

High-risk myelodysplastic syndrome. Myelodysplastic syndrome (MDS) is a clonal hematopoietic stem cell disorder characterized by evidence of dysplasia in two or more of the hematopoietic cell lines. Patients with these disorders suffer from refractory cytopenias predisposing them to the complications of marrow failure (infections, bleeding and fatigue) and have a predisposition to progress into acute leukemia (AML). The original FAB classification categorized these syndromes into five subtypes with differing morphologic features and prognoses. Prognosis in MDS varies according to FAB subtype, karyotype, patient age, percent blasts in the marrow and degree of cytopenia. Recently, the distinction between AML and MDS has become blurred secondary to the presence of several common features in the two disorders, such as the presence of common cytogenetic abnormalities, presence of dysplastic features in de novo AML, and the presence of very similar biologic and genetic features between AML arising in the older individuals and primary, secondary and therapy-induced MDS. Therefore, MDS is a part of the same disease continuum as AML and should be considered as a preleukemic disorder with variable rate of progression to AML. Indeed, most recently investigators have embarked upon treating patients on AML-related therapeutic regimens including combination chemotherapy and allogeneic transplantation. An alternative approach to assigning therapy is using a risk-based classification system (such as the International Prognostic Scoring System, IPSS) to facilitate clinical decision making. An overall IPSS score is highly predictive of median survival. A large number of agents have been evaluated in MDS ranging from androgens, corticosteroids, cytokines (such as G-CSF, GM-CSF, erythropoietin), Vitamin D, and retinoids in an attempt to induce differentiation in the dysplastic cell lines. None of these agents has demonstrated an improved outcome though the cytokines can improve single lineage cytopenias temporarily. Indeed, currently there is no standard therapy for the management of these disorders. As this disease is more common in the elderly population, use of agents able to induce differentiation with minimal toxicity is warranted. Patients with high-risk disease as predicted by the IPSS score are candidates for investigational treatment options as their life expectancy is otherwise limited.

Multiple myeloma. Multiple myeloma is among the more common haematopoietic cancers. There is a rapid increase in incidence with increasing age and a moderate excess at all ages in males. Geographical and racial differences play an important part in the incidence of myeloma. The disease is much more common in black populations than in Caucasians, and has a low incidence in Chinese. The roles of genetic background and environment are poorly defined.

For the diagnosis of multiple myeloma at least two of the following characteristics should be present: monoclonal immunoglobulin (paraprotein) in blood and/or urine, and bone-marrow infiltration by malignant plasma cells or osteolytic bone lesions. Multiple myeloma usually is preceded by an age-dependent premalignant tumor called monoclonal gammopathy of undetermined significance (MGUS), which is present in 1% of adults, progresses to malignant multiple myeloma at a rate of 1% per year. Multiple myeloma arises from a normal geminal-center B cell. At least 30-50% of malignant multiple myeloma appears to arise from the benign plasma-cell neoplasm MGUS. It does not always pass through a period of smouldering myeloma (stable intramedullary tumor content of greater than 10% with none of the malignant features of multiple myeloma). Initially, multiple myeloma is confined to the bone marrow, but with time the tumor can acquire the ability to grow in extramedullary locations. The transition of MGUS to intramedullary multiple myeloma is manifested by increased numbers of multiple myeloma cells at multiple foci, and also associated with angiogenesis and osteolytic bone destruction.

Numeric chromosomal abnormalities are present in virtually all multiple myelomas, and in most, if not all, cases of MGUS. Karyotypic complexity is thought to increase during tumor progression, although it has not been documented. Understanding how the karyotype correlates with disease severity is important because the detection of an abnormal karyotype correlates with poor prognosis. Translocations are common. The incidence of heavy-chain translocations increases with the stage of tumorigenesis. Most Ig translocations involve just three groups of genes: cyclins D1-3, MMSET and FGFR3, and two B-zip transcription factors (c-MAF and MAFB). Complex translocations dysregulate c-Myc as a late progression event that is associated with enhanced proliferation. c-Myc is rearranged in 15% of multiple myeloma representing all stages, but this fraction correlates with the severity of disease and is often heterogeneous among cells within the tumor. Studies that address the expression of c-Myc RNA and protein in multiple myeloma samples from patients are consistent with increased expression of c-Myc late in the disease. The temporal relationship between karyotypic abnormalities and IgH translocations is not understood, but it is possible that karyotypic instability is the initiating oncogenic event, whether they have an IgH translocation or not. Secondary translocations contribute to subsequent progression. The role of a possible tumor-suppressor gene on 13q remains enigmatic. However, progression from MGUS to myeloma is associated with activating mutations of RAS or FGFR3. This progression seems to flip a molecular switch that results in osteolytic bone lesions that are mediated by osteoclastogenesis, neoangiogenesis and enhanced growth of the myeloma clone. Further tumor progression, and especially extramedullary growth, is associated with increased proliferation, mutations of p53 and secondary translocations that dysregulate c-Myc.

Therapy for myeloma has relied predominantly on glucocorticoids, such as prednisone, which are nonspecific antiinflammatory agents, and alkylating agents, primarily melphalan. The latter seems to be more effective when a myeloablative dose is given along with an autologous stem-cell support. Neither therapy is curative and the median survival has remained fixed at about 3 years for the past decade. Although MGUS can be efficiently diagnosed by a simple blood test, it is not possible to prevent progression of even predict when progression to myeloma will occur. Clearly, there is a need for new drugs that may be useful for the control, treatment and/or cure of this disease.

Despite significant advances in the therapy of acute leukemias and lymphomas over the past several decades, treatment of relapsed and refractory AML and ALL, blast phase CML, relapsed and refractory high-grade lymphomas and high-risk MDS, as well as multiple myeloma, remains unsatisfactory. The need to identify new agents with antileukemic activity and reasonable safety profile to be incorporated into new regimens persists. In preclinical studies brusatol and bruceantin have demonstrated significant activity in various leukemic cell lines by inducing terminal differentiation and apoptosis, possibly mediated by down-regulation of c-Myc proteins. Bruceantin has been studied in phase I and II studies in patients with solid tumors (breast cancer, melanoma, and sarcoma) and a dose of 3.5 mg/m²/day for five days repeated in 3- to 4-week cycles has been found to be safe for clinical trials. Bruceantin also has demonstrated in vitro and in vivo activity in the treatment of multiple myeloma.

Investigators have searched for new cancer chemopreventative and chemotherapeutic agents by evaluating hundreds of plant extracts for a potentially active compounds. In this search for cancer chemopreventive and chemotherapeutic natural products, seeds of B. javanica were fractionated because an ethyl acetate extract of the seeds significantly induced cell differentiation with human promyelocytic leukemia (HL-60) cells. It was previously demonstrated that HL-60 cell differentiation is a valid system to assist in the discovery of potential cancer chemopreventive agents of natural origin. See N. Suh et al., Anticancer Res., 15, p. 233 (1995).

Bioassay-guided fractionation of the ethyl acetate extract of B. javanica using the HL-60 test system led to the isolation and identification of five active compounds including a lignan (guaiacyl-glycerol-β-O-6′-(2-methoxy)cinnamyl alcohol ether), three simaroubolides (brusatol, dehydrobrusatol, and yadanziolide C), and a terpenoid (blumenol A). Two further known compounds, cleomiscosin A and bruceoside B, also were isolated, but found to be inactive in the HL-60 test system. See L. Luyengi et al., Phytochemistry, 43, pp. 409-412 (1996).

Brusatol exhibited a potent induction of HL-60 cell differentiation, with an ED₅₀ of 0.006 μg/ml. Further, brusatol inhibits TPA-induced anchorage-independent growth of JB6 cells in a dose-dependent manner. Preneoplastic lesions also were inhibited by brusatol in the DMBA-induced mammary organ culture model with an ED₅₀ of 1 μg/ml. Yadanziolide C also was active in the HL-60 test system (ED₅₀=0.6 μg/ml), whereas bruceoside B was inactive. Due to a potential to induce HL-60 cell differentiation, to inhibit DMBA-induced mouse mammary lesions in organ culture, and to inhibit TPA-induced JB6 cell transformation, brusatol was considered as a candidate for cancer chemoprevention and chemotherapy. See N. Suh et al., 36th Annual Meeting of the American Association of Pharmacognosy, University of Mississippi, Oxford, Miss., Abstract P:107, July 23-27 (1995); and E. Mata-Greenwood et al., Proc. Am. Assoc. Cancer Res., 40, p. 127 (1999). However, researchers still searched for potent, nontoxic compounds capable of mediating desirable chemopreventive and chemotherapeutic activities.

### SUMMARY OF THE INVENTION

The present invention is directed to cancer chemopreventative and chemotherapeutic agents, compositions containing the agents, and methods of using the chemopreventative and chemotherapeutic agents to prevent and/or treat a cancer, like a leukemia or a lymphoma. In particular, the present invention is directed to compositions containing brusatol, bruceantin, glaucarubolone, and derivatives thereof, and use of the compositions in methods of cancer chemoprevention and chemotherapy. The invention also is directed to the use of brus-alone and glaucarubolone derivatives.

An important aspect of the present invention, therefore, is to provide a method and composition for preventing or treating a cancer using brusatol, bruceantin, glaucarubolone, or a derivative thereof.

Another aspect of the present invention is to overcome the problem of high mammalian toxicity associated with present cancer chemopreventative or chemotherapeutic agents by using a natural product-derived compound or a derivative thereof.

Still another aspect of the present invention is to overcome the problem of insufficient availability associated with synthetic anticancer agents by utilizing readily available, and naturally occurring, chemopreventative or chemotherapeutic agent or precursor.

Another important aspect of the present invention is to provide a drug composition containing brusatol, bruceantin, glaucarubolone, or a derivative thereof, and that can be administered to chemoprevent or treat cancers, and in particular, multiple myeloma.

Another aspect of the present invention is to provide chemopreventative or chemotherapeutic compositions having a potent antiproliferative effect with respect to promyelocytic leukemia HL-60 and other leukemic cells, e.g., the RPMI 8226 cell line for multiple myeloma, as defined by a low IC₅₀ value, and a low cytotoxic effect, as defined by a high IC₅₀ value.

These and other aspects of the present invention will become apparent from the following detailed description of the preferred embodiments of the invention.

### BRIEF DESCRIPTION OF THE DRAWINGS

FIGS. 1a and 1b are plots of NBT-positive cells (%) versus brusatol (ng/ml) for various cell lines;

FIG. 2a contains stains of cell treated and untreated with brusatol;

FIG. 2b contains a plot of benzidine-positive cells (%) versus brusatol (ng/ml) for various cell lines;

FIGS. 3a and 3b contain bar graphs for cell viability (%) and NBT-positive cells (%) for time of treatment;

FIG. 4 contains Western blots for various cell liner treated with brusatol and bruceantin;

FIG. 5 contains bar graphs of % cell growth relative to control in a hollow fiber test using 0.25 to 12.5 mg/kg brusatol;

FIG. 6 contains immunoblots for the expression of c-Myc and β-actin determined with RPMI 8226 cell lines and treated with brusatol and bruceantin;

FIG. 7 contains immunoblots for the expression of caspase-8, caspase-9, and PARP determined with RPMI 8226 cell lines and treated with bruceantin;

FIG. 8 contains plots for % apoptosis versus bruceantin (ng/ml) with and without caspase inhibitor;

FIG. 9 contains histograms showing the effect of bruceantin on mitochondrial membrane potential in the RPMI 8226 cell line;

FIG. 10 contains bar graphs for caspase 3/7 fold induction versus treatment time (hours);

FIGS. 11A and 11B, FIGS. 13A and 13B, and FIGS. 15A and 15B contain plots showing the effect of bruceantin on body weight (g) and on estimated tumor volume (mm³) in female rats, respectively; and

FIG. 12, FIG. 14, and FIG. 16 contain a plot of % volume versus days past inoculation showing the effect of bruceantin on tumor volume in 6-week-old females SCID mice having RPMI 8226 tumors.

### DETAILED DESCRIPTION OF THE PREFERRED EMBODIMENTS

The process of neoplastic cell growth can be depicted as a dysfunctional balance between control of cell proliferation, apoptosis, and terminal differentiation. In normal cells, activation of specific pathways leads to cellular differentiation, which typically is accompanied by cell growth arrest followed by apoptosis. In many cancers, like leukemias, genetic changes (e.g., chromosomal translocations, point mutations, gene amplifications or deletions) block the normal differentiation program. Conventional cytotoxic chemotherapy focuses on cell killing effects in order to achieve complete hematological remissions (i.e., less than 5% blasts). In the past few years, however, several nonconventional selective antileukemic agents have been developed that function by targeting molecules involved directly in the pathogenesis of the disease. For instance, all-trans-retinoic acid (ATRA) has revolutionized the treatment of acute promyelocytic leukemia (APL). Complete remissions are attained without marrow hypoplasia or exacerbation of fibrinolysis.

Although the mechanism of ATRA is still under investigation, it is known that binding with its natural receptor, RARα, results in the induction of granulocytic differentiation followed by apoptosis in APL-derived leukemic blasts. Another selective agent, CGP57148B, inhibits enhanced Abelson leukemia (ABL) tyrosine kinase activity resulting from the BCR/ABL fusion gene that is characteristic of leukemias with the t. Apoptosis is thereby induced selectively in these cases.

Some genes have been shown to be important in the development or malignancy of various types of leukemia and lymphoma, by inducing blockages in differentiation or apoptosis. Among them, c-Myc gene amplifications and translocations resulting in its deregulation have been noted, particularly in Burkitt's lymphoma and acute lymphoblastic leukemia (ALL). Studies using c-Myc knockout cell lines and c-Myc antisense RNA have shown that reducing c-Myc slows cell growth and induces differentiation in various cell lines. Moreover, regulation of c-Myc protein levels has proven to be an essential mode of action for various inducers of cellular differentiation.

Brusatol is a quassinoid, i.e., a type of degraded diterpenoid, obtained from Brucea species (Simaroubaceae). Brusatol and analogues are capable of inducing an array of biological responses including in vivo antiinflammatory and antileukemic effects with marine models. The major mechanism responsible for antineoplastic activity at the molecular level has been attributed to inhibition of protein synthesis. Such inhibition has been shown to occur via interference at the peptidyltransferase site, thus preventing peptide bond formation. Other cellular targets include inhibition of phosphoribosyl pyrophosphate aminotransferase of the de novo purine synthesis pathway and inhibition of DNA/RNA synthesis.

In order to assess toxicity, bruceantin (a structural analogue of brusatol) was evaluated in three separate phase I clinical trials in patients with various types of solid tumors. Hypotension, nausea, and vomiting were common side effects at higher doses, but hematologic toxicity was moderate to insignificant and manifested mainly as thrombocytopenia. Bruceantin then was tested in two separate phase II trials including adult patients with metastatic breast cancer and malignant melanoma. No objective tumor regressions were observed and clinical trials were terminated.

HL-60 cell differentiation activity was used as one marker of activity. This led to the identification of brusatol as a potent inducer of HL-60 cell differentiation. In order to test its potential efficacy as an antileukemic agent, the effect of brusatol with a panel of leukemic cells with representative chromosomal translocations and other gene mutations was evaluated. It was demonstrated that brusatol induces cell death events selectively in some cell lines, particularly those known to express wild-type p53, and induces terminal differentiation in the remaining cell lines. A significant finding was potent down-regulation of c-Myc oncoproteins; those cell lines expressing high levels of c-Myc oncoprotein were the most sensitive to brusatol-mediated effects. The decrease in c-Myc oncoprotein expression was due in part to transcriptional regulation, as shown by real-time RT-PCR, although the decrease in c-Myc transcript levels was less than the decrease of c-Myc protein levels. The potent down-regulation of c-Myc associated with strong cytotoxic and terminal cell differentiation events at physiologically achievable concentrations suggest this compound is a strong candidate for leukemia chemotherapy.

### MATERIALS AND METHODS

Materials

Brusatol was isolated from Brucea javanica and bruceantin was obtained from the NCI. 1α,25-Dihydroxyvitamin D₃ (VD₃) was supplied by Steroids, Ltd. (Chicago, Ill.), and 12-O-tetradecanoylphorbol-13-acetate (TPA) was purchased from Chemsyn Science Laboratories (Lenexa, Kans.). All other compounds were purchased from Sigma Chemical Co. (St. Louis, Mo.). Test compounds were dissolved in DMSO (dimethylsulfoxide) and stored at −20° C. Cell culture medium was obtained from Gibco BRL (Gaithesburg, Md.). [³H]Thymidine was obtained from Amersham Life Sciences (Arlington Heights, Ill.). Primary antibody for c-Myc (cat. No. OP10) was purchased from Oncogene (Cambridge, Mass.), and secondary antibody was from Amersham Life Sciences (Arlington Heights, Ill.). Primary antibody for β-actin was purchased from Sigma (St. Louis, Mo.), and all reagents utilized for real time RT-PCR were from Applied Biosystems (Foster City, Calif.).

Cell Culture

HL-60, K562, U937, Reh, Daudi, and RPMI 8226 cells were obtained from the American Type Culture Collection (Rockville, Md.). Kasumi-1, NB4, BV173, SUPB13, and RS4;11 cells were provided by the Section of Hematology/Oncology, University of Illinois College of Medicine, Chicago, Ill. All cell lines were maintained in suspension culture using RPMI 1640 medium supplemented with 10% heat-inactivated fetal bovine serum, 100 units of penicillin/ml and 100 μg of streptomycin/ml at 37° C. in a humidified atmosphere of 5% CO₂ in air. All cells were routinely tested for mycoplasma contamination.

Preparation of Normal Human Lymphocytes

Human blood (20 ml) was collected in heparinized sterile tubes and white blood cells were separated using Ficoll reagent (8 ml/5 ml blood diluted in 15 ml Hank's buffered solution). After centrifugation at low speed (1500 rpm) for 30 min, the white coat was removed and washed 3-times with Hank's buffered solution. The cell pellet was resuspended in RPMI 1640 medium supplemented with 10% FBS. This preparation contained >90% lymphocytes and <5% monocytes, as determined by Wright-Giemsa staining.

Cell Differentiation Assays

Cell lines were tested using a 4-day incubation protocol, unless otherwise specified. At the end of the incubation, cells were analyzed to determine the percentage exhibiting morphological, functional nitroblue tetrazolium (NBT) reduction, enzymatic nonspecific/specific esterase (NSE/SE) and cell surface markers of differentiated cells, as described below.

1) Cell morphology. Aliquots of the cell suspension (2×10⁵ cells/ml) were used to prepare cytospin smears which were stained with Wright-Giemsa. Morphological features of cellular differentiation (change in cytoplasmic pH, decrease in size, decrease of nuclear/cytoplasm ratio (or absence of nucleus), presence of specific granules or lysosomal vacuoles, lobulated nucleus) were monitored by light microscopy.

2) NBT/NSE/SE. Evaluation of NBT reduction was used to assess the ability of sample-treated cells to produce superoxide when challenged with TPA. A 1:1 (v/v) mixture of a cell suspension (10⁶ cells) and TPA/NBT solution (2 mg/ml NBT and 1 μg/ml TPA in phosphate buffer saline (PBS)) was incubated for 1 h at 37° C. Then, cells were smeared on glass slides, and counterstained with 0.3% (w/v) safranin O in methanol. Positive cells reduce NBT yielding intracellular black-blue formazan deposits. NSE/SE are monocytic/granulocytic esterases that can be visualized by cytochemical staining using commercially available kits (α-Naphthyl Acetate Esterase and Naphthol As-D Chloroacetate Esterase kits, Sigma Chemical Co., St. Louis, Mo.). Positive-stained cells were quantified by microscopic examination of >200 cells. Results were expressed as a percentage of positive cells.

3) Determination of cell surface antigen by flow cytometry Cells (10⁶) were washed with PBS and then incubated for 30 min at room temperature with respective monoclonal antibodies, washed with 20 volumes of diluent (PBS with 0.1% sodium azide and 1% BSA), and resuspended in 0.5 ml of fresh diluent for evaluation. Necrotic cells were excluded from the analysis by propidium iodide (PI) staining. The following mAbs (Sigma, St. Louis, Mo.) were used to assess the maturation level of myeloid cell lines: antiCD15 (Leu Ml), antiCD-11b (OKM1), antiCD14 and antiCD13. The following mabs (Sigma, St. Louis, Mo.) were used to assess the maturation level of lymphocytic cell lines: antiCD20, antiHLA-DR and antikappa light chain.

Cell Growth and Viability Assays

Cellular viability was monitored by Trypan blue exclusion. Inhibition of [³H]thymidine incorporation into DNA was determined to assess the level of cell proliferation as well as DNA synthesis inhibition. Cells were treated with test samples for four days and then placed into 96-well plates (100 μl) and treated with [³H]thymidine (0.5 μCi/ml, 65 Ci/mmol) for 18 h at 37° C. in a 5% CO₂ incubator. Cells then were collected on glass fiber filters using a TOMTEC Harvester 96®. The filters were counted using a Microbeta™ liquid scintillation counter (Wallac, Turku, Finland) with scintillation fluid. Finally, the percentage of [³H]thymidine incorporation per 10⁶ cells was calculated by dividing the dpm of sample-treated cells by the dpm of DMSO-treated cells.

Analysis of DNA Content with Flow Cytometry

About 10⁶ cells from each sample were collected and washed twice with ice-cold PBS, fixed in 70% ethanol, and stored at 4° C. until analysis. The cells were stained with PI (50 μg/ml), treated with DNase-free RNase (10 μg/ml), and subjected to DNA content analysis using an EPICS Coulter flow cytometer. At least 10,000 cells were counted for each sample. The percentage of apoptotic cells was calculated by measuring the area under the subdiploid (DNA <2 N) peak in the plot of cell number against cellular DNA content.

Immunoblotting

The expression of c-Myc was assessed by immunoblots as previously described. In brief, cells (10⁶) were treated and harvested at various time intervals, and whole-cell pellets were lysed with detergent lysis buffer (1 ml/10⁷ cells, 50 mM Tris-HCl buffer, pH 8.0, 150 mM NaCl, 1 mM DTT, 0.5 mM EDTA, 1% Nonidet® P40, 0.5% sodium deoxycholate, 0.1% sodium dodecylsulfate, 100 μg/ml phenylmethyl-sulfonoyl fluoride, 1 μg/ml aprotinin, 2 μg/ml leupeptin and 100 μM sodium vanadate) to obtain protein lysates. Protein concentrations were quantified using a bicinchoninic acid kit. Since c-Myc is a labile protein, cell lysates were not frozen, but stored at 4° C., until all protein lysates were prepared for a particular cell line, and then western blots were performed immediately. Total protein (30 mg) was separated by 10% SDS-PAGE, electroblotted to PVDF membranes, and blocked over-night with 5% nonfat dry milk. The membrane was incubated with a solution of the primary antibody (2.5 μg/ml), prepared in 1% blocking solution, for 2 h at room temperature, washed three-times for 15 min with PBS-T (PBS with 0.1%, v/v, Tween 20), and incubated with a 1:2500 dilution of horseradish peroxidase-conjugated secondary antibody for 30 min at 37° C. Blots were again washed three-times for 10 min each in PBS-T and developed by enhanced chemi-luminescence (Amersham). Membranes were exposed to Kodak Biomax film and the resulting film was analyzed using Kodak 1D Image Analysis software. Membranes were then stripped and reprobed for the quantification of b-actin.

RT-PCR Analysis

RNA was extracted from 106 cells using TRIZOL® Reagent (Life Technologies). Following isopropanol precipitation, the pellet was washed in 75% aqueous ethanol and the RNA was dissolved in 25 ml of diethyl pyrocarbonate (DEPC)-treated distilled water. Subsequently, the samples were stored at −80° C. RNA quantitation was performed by UV measurement at 260 nm. The CDNA synthesis was performed in a total volume of 10 ml, containing lx TaqManO RT buffer, 5.5 mM MgCl2, 2 mM dNTPs mixture, 2.5 mM random hexamers, 4 U RNase inhibitor, 12.5 U MultiScriber RT (Perkin Elmer/Applied Biosystems) and 0.2 mg of RNA. The reaction was performed for 10 min at 25° C., followed by 48° C. for 30 min and a 5 min incubation step at 95° C. After the reaction, 10 ml of DEPC-treated distilled water was added to each sample and 1 ml was used for each PCR.

The PCR and subsequent analyses were performed in the GeneAmp 5700 Sequence Detection System (Applied Biosystems). Real-time quantitation was performed using the TaqMan technology of Applied Biosystems (Foster City, Calif., USA). c-Myc primers and probe sequences (5′ to 3′) were as follows: CGTCTCCACACATCAGCACAA, TCTTGGCAGCAGGATAGTCCTT and TACGCAGCGCCTCCCTCCACTC (Applied Biosystems).

PCR reactions were performed in triplicate. The PCR reaction mixture contained 300 nM of both primers, 150 nM TaqMan probe, and 1× TaqMan Universal Master Mix (Applied Biosystems). The reactions first were incubated at 50° C. for 2 minutes, followed by 10 min at 95° C. The PCR itself consisted of 40 cycles with 15 seconds at 95° C. and 1 minute at 60° C. each. The fluorescence signal was measured during the last 30 seconds of the annealing/extension phase. After the PCR, a fluorescence threshold value was set and threshold cycle (Ct) values were determined, i.e., the fractional cycle at which the fluorescence signal reached this threshold. These values were used for further calculations.

β-Actin (TaqMan PDAR control, Applied Bio-systems) was used as an endogenous reference to correct for any differences in the amount of total RNA used for a reaction and to compensate for different levels of inhibition during reverse transcription of RNA into cDNA. c-Myc and β-actin expression were related to a standard curve derived from a serial dilution of K562 cDNA with dH₂O. Also, c-Myc and β-actin quantities were expressed in terms of ng of K562 RNA yielding the same level of expression. Subsequently, normalization was achieved by dividing the expression level of c-Myc by the β-actin expression level. Finally, results were expressed as a percentage, where the level of c-Myc observed in the DMSO-treated samples was considered as 100%.

### RESULTS

Cytotoxic and Antiproliferative Effects of Brusatol on Normal Human Lymphocytes and Leukemic Cells

A panel of eleven leukemic cell lines showing various chromosomal aberrations (Table 1) was selected, and the effects of brusatol and bruceantin on cell viability and proliferation were tested. Evaluation of viability using the Trypan blue exclusion method demonstrated that brusatol was preferentially cytotoxic to the NB4, U937, BV173, SUPB13, RS4;11, Daudi and DHL-6 cell lines, showing IC₅₀ values of less than 25 ng/ml (Table 1). On the other hand, HL-60, Kasumi-1, and Reh cell lines showed increased resistance to cytotoxic effects with IC₅₀ values in the range of 50-100 ng/ml. K562 and normal lymphocytic cells (stimulated with concanavalin A) were the least sensitive of all cells tested, demonstrating approximately 90% viability after 4 days of treatment with 100 ng/ml of brusatol (Table 1). Bruceantin, which differs from brusatol by two methyl groups in the ester side chain at C-15, was more potent than brusatol in all cell lines tested (Table 1). There was no obvious correlation between cytotoxic activity and a particular chromosomal aberration.

TABLE 1
In vitro effects of brusatol and bruceantin on
cell growth and proliferation of various established
leukemic cell lines and peripheral human lymphocytes

| | | | Antiproliferative | | |
|---|---|---|---|---|---|
| | Cytotoxicity^{a} | | Activity^{b} | | |
| Cell | (IC₅₀, ng/ml) | | (IC₅₀, ng/ml) | | Cell Line |
| Line | Brusatol | Bruceantin | Brusatol | Bruceantin | Characteristics |
| HL-60 | 50 ± 0.9 | 20 ± 0.8 | 25 ± 0.5 | 20 ± 0.1 | AML, c-myc |
| | | | | | amplification³³ |
| K562 | >100 | >50 | 10 ± 0.2 | 6 ± 0.1 | CML, t(9;22) |
| | | | | | c-myc |
| | | | | | disregulation^{34,16} |
| Kasumi-1 | 75 ± 5.1 | 39 ± 4.5 | 7 ± 0.6 | 3.2 ± 0.1 | AML, t(8;21): |
| | | | | | ETO/AMLI³⁵ |
| NB4 | 15 ± 0.2 | 5 ± 0.3 | No effect | No effect | AML, t(15,17): |
| | | | | | PML/RARα³⁶ |
| U937 | 12.5 ± 1.1 | 7 ± 0.1 | No effect | No effect | AML, c-myc |
| | | | | | disregulation³⁷ |
| BV173 | 5 ± 1.4 | 2.1 ± 0.2 | No effect | No effect | CML, t(9;22): |
| | | | | | BCR/ABL³⁸ |
| SUPB13 | 20 ± 1.2 | 15 ± 0.5 | 37 ± 1.9 | 10 ± 0.6 | ALL, t(9;22)³⁹ |
| RS4;11 | 25 ± 2 | 10.2 ± 0.4 | 12.5 ± 0.6 | 5.4 ± 0.9 | ALL, t(4;11)⁴⁰ |
| Reh | 100 ± 5.4 | 45 ± 1.1 | 37 ± 5.6 | 12.5 ± 0.8 | ALL, t(12;21): |
| | | | | | TEL/AMLI⁴¹ |
| Daudi | 5 ± 0.3 | 1.7 ± 0.1 | No effect | No effect | Burkitt lymphoma, |
| | | | | | t(8;14):c- |
| | | | | | MYC/IgH^{8,9} |
| DHL-6 | 5 ± 0.5 | 2.0 ± 0.1 | No effect | No effect | Diffuse |
| | | | | | histiocytic |
| | | | | | lymphoma |
| | | | | | t(14;18); |
| | | | | | BCL-2/IgH⁴² |
| Peripheral | >100 | >50 | 7.5 ± 0.3 | 3 ± 0.3 | |
| Human | | | | | |
| Lymphocytes | | | | | |

Cells were incubated with various concentrations of brusatol or bruceantin for four days and normal human lymphocytes were allowed to grow three days in the presence of concanavalin A (0.4 μg/ml) and various concentrations of brusatol.
^{a}Viability was determined by Trypan blue exclusion in duplicate samples. Control viabilities did not decrease from 90%. Results are expressed as median inhibitory cell concentrations of two independent experiments (± standard deviations).
^{b}Proliferation was assayed by [³H]thymidine incorporation with quadruplicate samples, as described under “Materials and methods.” Results are shown as median inhibitory cell concentrations of two independent experiments (± standard deviations).

The effects of brusatol on proliferation of normal human lymphocytes or leukemic cells was examined by incorporation of [³H]thymidine into DNA over an 18 h incubation period, subsequent to exposure to various concentrations of brusatol for 4 days. Brusatol inhibited the proliferation of normal human lymphocytes, HL-60, K562, Kasumi-1, SUPB13, RS4;11, and Reh cells in a dose-dependent manner (Table 1). Interestingly, these cell lines represent those that were most resistant to brusatol-mediated cytotoxicity, while the compound actually increased the amount of radioactive precursor incorporation in some cytotoxic-sensitive cell lines NB4, U937, BV173, and Daudi (data not shown).

In accordance with [³H]thymidine incorporation data, brusatol (25 ng/ml) significantly induced G₁ arrest (with concomitant decreases in S and/or G₂/M phases) in asynchronious HL-60, K562, Kasumi-1, BV173, SUPB13, and Reh cells (Table 2), and the G₁ block was complete at 72 h using a higher dose of 100 ng/ml (data not shown). NB4 and BV173 cells showed sub-G₁ peaks characteristic of apoptosis, while U937 and RS4;11 cells did not (Table 2), although loss of viability (as determined by Trypan blue exclusion) was similar for all four cell lines. Interestingly, U937 and RS4;11 cells showed a decrease in the G₁ phase and a significant increase in the S phase, characteristic of metabolic arrest.

TABLE 2
Cell cycle effects of brusatol
with various leukemic cell lines

| Cell line | Control, 24 h | Brusatol (25 ng/ml), 24 h |
|---|---|---|
| HL-60 | G₁ = 39.3 ± 1.3 | G₁ = 68.7 ± 0.9* |
| | S = 50.8 ± 1.6 | S = 13.0 ± 1.0** |
| | G₂/M = 9.9 ± 2.8 | G₂/M = 18.3 ± 1.1 |
| K562 | G₁ = 31.2 ± 1.8 | G₁ = 46.2 ± 0.6* |
| | S = 57.1 ± 2.1 | S = 48.2 ± 0.3 |
| | G₂/M = 11.7 ± 0.4 | G₂/M = 5.60.4* |
| Kasumi-1 | G₁ = 57.0 ± 0.4 | G₁ = 77.4 ± 1.8* |
| | S = 38.3 ± 1.1 | S = 18.1 ± 2.1* |
| | G₂/M = 4.7 ± 0.6 | G₂/M = 4.5 ± 0.2 |
| NB4 | G₁ = 40.9 ± 1.8 | G₁ = 45.3 ± 0.8 |
| | S = 53.0 ± 2.1 | S = 47.4 ± 0.1 |
| | G₂/M = 6.1 ± 0.1 | G₂/M = 7.3 ± 0.7 |
| | Ap = 11.5 ± 0.5 | Ap = 37.2 ± 0.8* |
| U937 | G₁ = 50.0 ± 0.1 | G₁ = 45.4 ± 1.8 |
| | S = 42.0 ± 0.5 | S = 46.6 ± 0.2* |
| | G₂/M = 8.0 ± 0.6 | G₂/M = 8.0 ± 1.6 |
| BV173 | G₁ = 47.7 ± 1.0 | G₁ = 57.0 ± 4* |
| | S = 43.3 ± 2.7 | S = 35.9 ± 0.6 |
| | G₂/M = 9.0 ± 1.6 | G₂/M = 7.1 ± 0.1 |
| | Ap = 0 | Ap = 15.6 ± 0.2* |
| SUPB13 | G₁ = 49.8 ± 1.6 | G₁ = 61.9 ± 2.1* |
| | S = 37.8 ± 0.6 | S = 29.4 ± 1.8 |
| | G₂/M = 12.4 ± 1.0 | G₂/M = 8.7 ± 0.3 |
| RS4,11 | G₁ = 58.6 ± 0.7 | G₁ = 50.9 ± 0.4* |
| | S = 3917 ± 0.3 | S = 45.7 ± 0.2** |
| | G₂/M = 1.7 ± 0.7 | G₂/M = 3.4 ± 0.1 |
| Reh | G₁ = 57 ± 1.0 | G₁ = 78.8 ± 1.2* |
| | S = 37.7 ± 0.5 | S = 18.0 ± 0.6** |
| | G₂/M = 5.3 ± 1.5 | G₂/M = 3.2 ± 0.4 |

Cells were treated with solvent or brusatol (25 ng/ml) for 24 hours, fixed in ethanol and stained with PI for flow cytometric analysis as described under “Materials and methods.” At least 10,000 cells were counted. Results are presented as the mean of triplicate samples (± standard deviation).
G₁ = Gap1, S = Synthesis, G₂/M = Gap2 + Mitosis, Ap = sub-G₁ apoptotic peak.
*Significantly different from control values, determined by Student's t-test (p < 0.05).
**Significantly different from control values, determined by Student's t-test (p < 0.005).

Induction of Differentiation by Brusatol with Various Myeloid and Lymphoblastic Cell Lines

Studies demonstrated that brusatol was able to induce differentiation of HL-60 cells in a concentration-dependent fashion. In the current study, cells were treated with various concentrations of brusatol for 4 days, then harvested for evaluation of functional, enzymatic and cell membrane markers of differentiation.

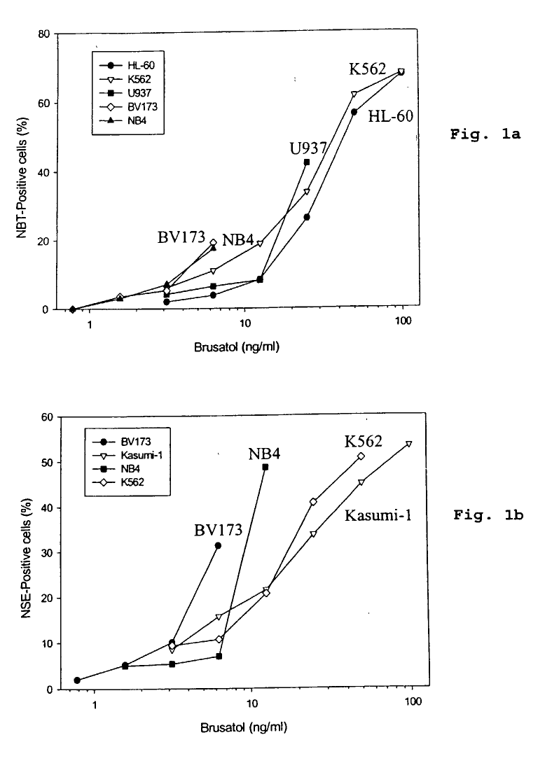

FIG. 1 shows that Brusatol induces monocyte-like characteristics in various acute and chronic myeloid leukemic cells. Concentration-dependent effect of brusatol on: (a) NBT-reduction (monocyte/granulocyte marker) of HL-60, K562, NB4, U937, and BV173 and (b) NSE expression (monocyte marker) in K562, Kasumi-1, NB4, and BV173 cells, respectively. Data points are the mean of duplicate samples.

Analysis of NBT-reduction for evaluation of superoxide formation demonstrated myeloid maturation in five cell lines (HL-60, K562, NB4, U937 and BV173). The effect was dose-dependent, as shown in FIG. 1a. Peak inductions of 75% were observed in HL-60 and K562 cells. In addition, brusatol up-regulated the expression of NSE (a monocytic marker) in K562, Kasumi-1 and NB4 by approximately 50%, and in BV173 cells by approximately 35% (FIG. 1b).

Membrane phenotype using flow cytometry with a set of four myeloid markers (CD11b, CD13, CD14, and CD15) also was analyzed. Brusatol up-regulated CD11b in HL-60 and U937 cells, CD13 in HL-60, NB4 and U937 cells, and CD14 only in U937 cells, and down-regulated CD15 in HL-60, K562, NB4, U937 and RS4;11 cells (Table 3). Thus, it was noted that brusatol induced a pattern of expression similar to that produced by macrophage inducers, with down-regulation of CD15 (granulocytic marker) and up-regulation of CD13 and CD11b (granulocytic/monocytic markers) in HL-60 and U937 cells (Table 3).

TABLE 3
Effect of brusatol on the membrane
phenotype of myeloid cell lines

| Cell type | | | | |
|---|---|---|---|---|
| and | | | | |
| treatment | CD11b | CD13 | CD14 | CD15 |
| HL-60 cells | | | | |
| Control | 1.1 ± 0.1 | 33.5 ± 3.5 | 1.0 | 362.4 ± 12.1 |
| Brusatol | 2.2 ± 0.5* | 266.1 ± 55.7* | 1.0 | 177.1 ± 8.7* |
| 25 ng/ml | | | | |
| K562 cells | | | | |
| Control | 1.3 ± 0.1 | 2.2 ± 1.3 | 1.0 | 19.1 ± 5.3 |
| Brusatol | 1.0 ± 0.1 | 1.3 ± 0.2 | 1.0 | 4.6 ± 1.1* |
| 25 ng/ml | | | | |
| NB4 cells | | | | |
| Control | 1.2 ± 0.3 | 58.8 ± 10.6 | 1.7 | 35.5 ± 9.5 |
| Brusatol | 1.2 ± 0.5 | 156.1 ± 54.3* | 1.3 | 18.5 ± 0.4* |
| 10 ng/ml | | | | |
| U937 cells | | | | |
| Control | 1.6 ± 0.1 | 38.6 ± 1.0 | 6.3 | 57.0 ± 9.5 |
| Brusatol | 2.5 ± 0.2* | 55.7 ± 1.1* | 9.6 | 28.5 ± 4.3* |
| 12.5 ng/ml | | | | |
| RS4;11 cells | | | | |
| Control | 1.4 ± 0.05 | 1.4 ± 0.07 | Not tested | 391.8 ± 90.5 |
| Brusatol | 1.2 ± 0.1 | 1.3 ± 0.2 | Not tested | 151.9 ± 42.1* |
| 25 ng/ml | | | | |

Cells were induced to differentiate with the indicated concentrations of brusatol using a 4-day protocol and then analyzed for membrane markers of differentiation as described under “Materials and methods.” Results are expressed as the specific mean fluorescence intensity (ratio of antigen antibody fluorescence over isotype antibody fluorescence), and represents the mean of two independent studies.
Kasumi-1 and BV173 did not show any changes between the control and treated cells.
*Significantly different from control values, determined by Student's t-test (p < 0.05).

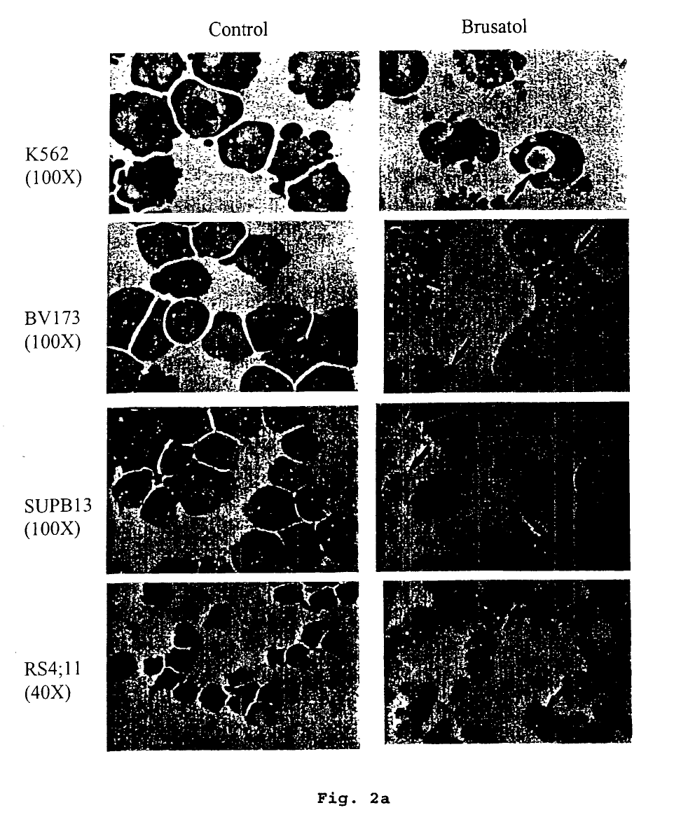

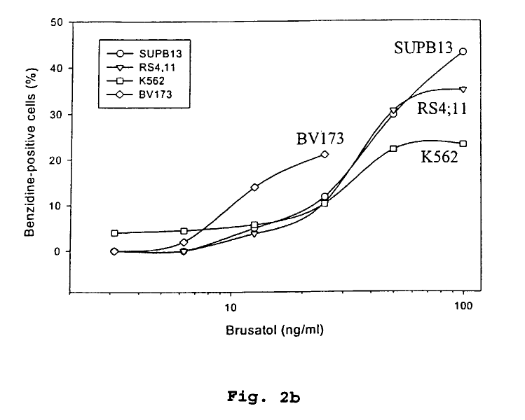

FIG. 2 shows that brusatol induces erythrocytic differentiation in chronic myeloid cell lines K562 and BV173 and acute lymphoblastic SUPB13 and RS4;11 cell lines. In FIG. 2a, morphological changes characteristic of erythroid differentiation were visualized by Wright-Giemsa staining for K562, BV173, SUPB13, and RS4;11 cells. Control cells and brusatol (25 ng/ml for K562 and SUPB13 and 5 ng/ml for BV173 and RS4;11)-treated cells were harvested at day 4 of incubation; differentiated cells are shown with an arrow. In FIG. 2b, concentration-dependent effect of brusatol on hemoglobin expression of CML and ALL cell lines. K562, BV173, SUPB13, and RS4;11 cells were incubated with varying concentrations of brusatol for four days, then analyzed for expression of hemoglobin using the benzidine staining method. Data points are the mean of duplicate samples.

It was of interest to note morpholoG₁cal changes characteristic of erythroid differentiation in two lymphoblastic cell lines (SUPB13 and RS4;11), as was shown for CML cell lines K562 and BV173 (i.e., smaller cells devoid of nuclei with a pinkish-bluish cytoplasm, FIG. 2a). Erythrophagocytosis by adjacent cells is also evident in some of the cell lines undergoing erythroid differentiation (FIG. 2a). This finding was supported by the production of hemoglobin in these cells, as shown by benzidine staining (FIG. 2b). Hemoglobin was up-regulated dose-dependently in SUPB13 and RS4;11 cells, as well as the CML cell lines K562 and BV173 (FIG. 2b).

Finally, various membrane markers of B-lymphocyte maturation (i.e., CD20, superficial light chain kappa and HLA-DR) in SUPB13, RS4;11, Reh, Daudi and DHL-6 were analyzed. Few changes were observed, but brusatol induced a small increase in CD20 with DHL-6 cells (197.5 control vs. 225.3 brusatol (5 ng/ml), specific mean fluorescence intensity), and a larger increase of HLA-DR in Daudi cells (92.5 control vs. 244.9 brusatol (10 ng/ml), specific mean fluorescence intensity). These preliminary data suggest brusatol enhances B-cell maturation.

Irreversibility of Brusatol Effects on Differentiation or Cell Death of Leukemic Cells

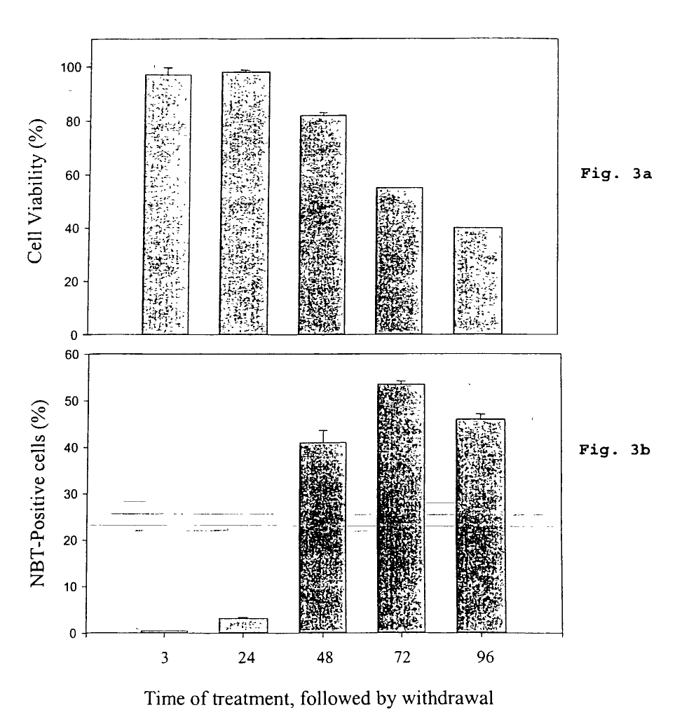

The irreversibility of brusatol effects on growth and differentiation of HL-60 cells was tested using withdrawal assays during a 4-day experiment. Withdrawal of brusatol after 48 h of exposure resulted in the induction of 41% of cells to differentiate (compared to 46% without withdrawal), while maintaining cellular viability higher than 80% and the same cell density (0.21×10⁶) as time zero (FIG. 3).

FIG. 3 shows commitment toward differentiation of HL-60 cells is obtained at 48 h of exposure to brusatol. The assay lasted for 4 days (96 h), then cells were analyzed for viability and differentiation markers. HL-60 cells were treated with 12.5 ng/ml of brusatol which was withdrawn after the indicated time intervals, and cells were resuspended in fresh complete media for the remaining time. Results are shown as the mean of duplicate samples (± standard deviation).

The percentage of cells induced to maturate is similar for time exposures of 48, 72 or 96 h, indicating there is no further need for the presence of the compound after 48 h, where cells have become committed to differentiate. However, the viability percentages were greatly reduced with increasing time of exposure to the drug (82% at 48 h; 56% at 72 h; 45% at 96 h), indicating the cytotoxic effect is cumulative (FIG. 3). Therefore, when comparing the concentration required to induce 50% of cells to differentiate with the concentration needed to kill 50% of cells using 4-day or 2-day exposure protocols, a 10-fold increase in selectivity was observed when the 2-day protocol was used (differentiation induction ED₅₀=17.5 ng/ml for both protocols; cytotoxic IC₅₀=25 ng/ml for a 4-day protocol and 250 ng/ml for a 2 day-protocol). Similar effects of withdrawing brusatol were observed in other cell lines, such as K562 and SUPB13, where commitment toward differentiation was obtained with 48 h of exposure to brusatol (data not shown). Withdrawal studies also demonstrated that 48 h of brusatol (25 ng/ml) treatment is sufficient to induce 100% cytotoxicity in NB4, Daudi, and DHL-6 cells, but not in the remaining cell lines (data not shown).

Brusatol Down-Regulates c-Myc

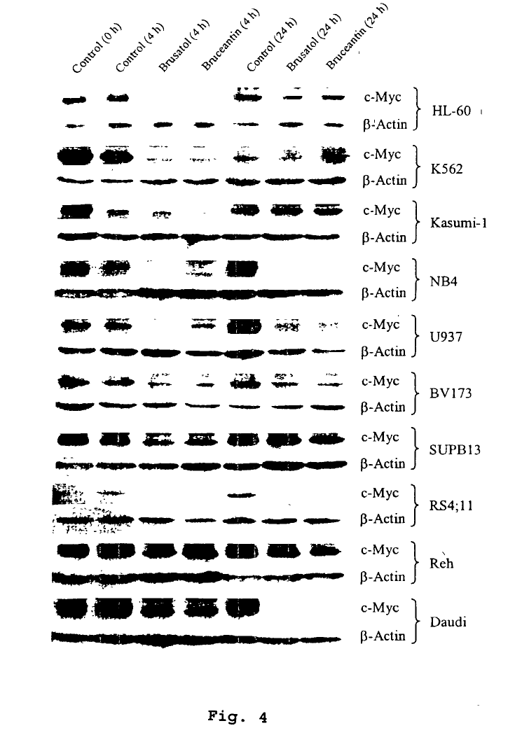

FIG. 4 shows that brusatol down-regulates c-Myc expression. Cells were treated with solvent (0.1% v/v DMSO, control), brusatol (25 ng/ml), or bruceantin (10 ng/ml) for 4 or 24 h, then analyzed by western blotting. Membranes were probed for c-Myc, and then stripped and probed for β-actin as an internal control. Densitometric analyses are summarized in Table 4.

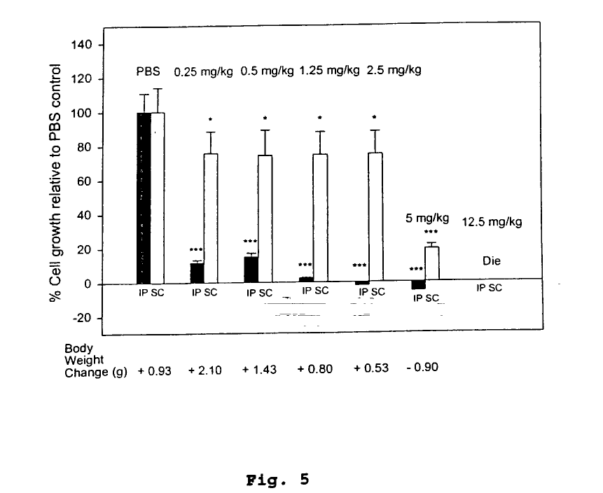

Because c-Myc deregulation is involved in blockage of differentiation, increased apoptosis and proliferation, the status of c-Myc in ten cell lines after a short exposure (4 or 24 h) to brusatol (25 ng/ml) or bruceantin (10 ng/ml) was analyzed. The level of c-Myc protein was high in control samples of HL-60, K562, Kasumi-1, SUPB13, Reh, and Daudi cells (FIG. 4). Moderate levels of c-Myc protein were observed in NB4, U937, BV173, and RS4;11 cells. Brusatol and bruceantin induced down-regulation of c-Myc protein levels in all cell lines, but greatest reduction occurred in HL-60, K562, NB4, U937, BV173, RS4;11, and Daudi cells (FIG. 5, Table 4). In contrast, c-Myc protein levels in Kasumi-1, SUPB13, and Reh cells were reduced to a lesser extent when treated with brusatol (FIG. 5, Table 4). Cytotoxic-sensitive cell lines NB4, U937, BV173, RS4;11 and Daudi cells showed marked decreases of c-Myc at 24 h, while those cell lines that manifested terminal differentiation (HL-60, K562 and SUPB13) showed the lowest levels of c-Myc protein at 4 hours. Interestingly, brusatol also down-regulated c-Myc expression in normal human lymphocytes, although control levels were low (data not shown).

Analysis of c-Myc mRNA using real time RT-PCR revealed that brusatol and bruceantin produced minor effects on the transcriptional regulation of c-Myc in those cell lines where protein expression was markedly reduced (Tables 4 and 5). For example, a 4 h treatment with brusatol induced a decrease in c-Myc mRNA levels by about 40 and about 50% in K562 and HL-60 cells, respectively. However, c-Myc protein levels were decreased by 94 and 100%, respectively. It is important to note that both protein and mRNA evaluations were performed in a parallel fashion, therefore avoiding experimental errors due to compound stability and cell line senescence.

These data suggest that brusatol and bruceantin are affecting translational regulation of c-Myc expression. Interestingly, the opposite effect was observed in Kasumi-1 and SUPB13 cells, were c-Myc transcript levels were significantly reduced, but c-Myc protein expression was similar to control (solvent-treated) samples (Tables 4 and 5).

TABLE 4
Effect of brusatol and bruceantin on c-Myc
oncoprotein expression in various leukemic cell lines

| | 4 hours | | 24 hours | |
|---|---|---|---|---|
| | Brusatol | Bruceantin | Brusatol | Bruceantin |
| Cell line | (25 ng/ml) | (10 ng/ml) | (25 ng/ml) | (10 ng/ml) |
| HL-60 | 0 | 3 | 17 | 57 |
| K562 | 6 | 2 | 13 | 12 |
| Kasumi-1 | 66 | 70 | 112 | 87 |
| NB4 | 0 | 0 | 0 | 0 |
| U937 | 5 | 40 | 12 | 44 |
| BV173 | 22 | 28 | 41 | 12 |
| SUPB13 | 52 | 42 | 41 | 63 |
| RS4;11 | 20 | 140 | 10 | 6 |
| Reh | 66 | 44 | 32 | 40 |
| Daudi | 79 | 67 | 10 | 2 |

Cells were treated with brusatol (25 ng/ml) or bruceantin (10 ng/ml) for 4 or 24 hours, and then analyzed for c-Myc and β-actin expression using Western blotting techniques. Results are expressed as percentage of c-Myc oncoprotein of control (0.1%, v/v, DMSO treated cells) values. Results were normalized relative to β-actin. Bands are shown in FIG. 5.

TABLE 5
Effect of brusatol and bruceantin on c-myc
mRNA levels in various leukemic cell lines

| | 4 hours | | 24 hours | |
|---|---|---|---|---|
| | Brusatol | Bruceantin | Brusatol | Bruceantin |
| Cell line | (25 ng/ml) | (10 ng/ml) | (25 ng/ml) | (10 ng/ml) |
| HL-60 | 54 ± 2* | 76 ± 9* | 11 ± 2* | 16 ± 0* |
| K562 | 62 ± 4* | 55 ± 2* | 45 ± 6* | 33 ± 2* |
| Kasumi-1 | 27 ± 5* | 20 ± 2* | 31 ± 3* | 15 ± 2* |
| NB4 | 34 ± 4* | 27 ± 4* | 24 ± 3* | 85 ± 5* |
| U937 | 103 ± 1 | 86 ± 1 | 19 ± 1* | 19 ± 2* |
| BV173 | 78 ± 12* | 125 ± 11* | 45 ± 0* | 67 ± 9* |
| SUPB13 | 27 ± 3* | 38 ± 2* | 24 ± 1* | 24 ± 2* |
| RS4;11 | 54 ± 5* | 68 ± 3 | 37 ± 2* | 31 ± 8* |
| Reh | 87 ± 2 | 68 ± 5* | 38 ± 3* | 42 ± 6* |
| Daudi | 100 ± 7 | 134 ± 17 | 84 ± 1* | 70 ± 5* |

Cells were treated with brusatol (25 ng/ml) or bruceantin (10 ng/ml) for 4 or 24 hours then analyzed for c-myc and β-actin mRNA using real time RT-PCR. Results are shown as percentage of c-myc mRNA expression of control (0.1%, v/v, DMSO) values. Results are the mean of triplicate samples (± standard deviation) . c-Myc mRNA values were normalized relative to β-actin.
*Significantly different from control values, determined by Student's t-test (p < 0.05).

As a preliminary test of in vivo efficacy, a hollow fiber study was performed. The in vivo hollow fiber test was performed using literature procedures with some modifications (see M. G. Hollingshead et al., Life Sic., 57, pages 131-141 (1995)). HL-60 cells were cultured in RPMI 1640 medium and collected by centrifugation and resuspended in conditioned medium at a concentration of 2.5×10⁶ cells/ml. Fibers filled with cells were incubated in 6-well plates overnight at 37° C. in a 5% CO₂ atmosphere. Female athymic NCr nu/nu mice at 5-6 weeks of age were obtained from Frederick Cancer Research Facility. Each mouse hosted up to 6 fibers, which were cultured in two physiologic compartments. For intraperitoneal implants, a small incision was made through the skin and musculature of the dorsal abdominal wall, the fiber samples were inserted into the peritoneal cavity in a cranio-caudal direction, and the incision was closed with skin staples. For subcutaneous implants, a small skin incision was made at the nape of the neck to allow insertion of an 11-gauge tumor implant trocar. The trocar, containing the hollow fiber samples, was inserted caudally through the subcutaneous tissues and fibers were deposited during withdrawal of the trocar. The incision was closed with a skin staple.

In preliminary studies, cell growth was assessed with fibers containing various cell densities. As a result, a cell density of 2.5×10⁶ cells/ml was found to be suitable for drug studies for HL-60 cells. For treatment protocols, brusatol was dissolved in PBS. Mice were randomized into 7 groups: PBS vehicle control group (6 mice per group); 0.25, 0.5, 1.25, 2.5, 5, 12.5 mg/kg of brusatol (3 mice per group). Test compound brusatol was administered once daily by intraperitoneal injection from day 3-6 after implantation. Body weights were measured daily.

On day 7, mice were sacrificed and fibers were retrieved. The fibers were placed into 6-well plates, each well containing 2 ml of fresh, pre-warmed culture medium and allowed equilibrating for 30 minutes at 37° C. To define the viable cell mass contained within the intact hollow fibers; an MTT dye conversion assay was used. Briefly, 1 ml of prewarmed culture medium containing 1 mg MTT/ml was added to each dish. After incubating at 37° C. for 4 hours, the culture medium was aspirated and the samples were washed twice with normal saline containing 2.5% protamine sulfate solution by overnight incubation at 4° C. To assess the optical density of the samples, the fibers were transferred to 24-well plates, cut in half, and allowed to dry overnight. The formazan was extracted from each sample with DMSO (250 μl/well) for 4 hours at room temperature on a rotation platform. Aliquots (150 μl) of extracted MTT formazan were transferred to individual wells of 96-well plates and assessed for optical density at a wavelength of 540 nm. The effect of the treatment regimen was determined by the net growth percentage of the cells relative to change in both weight.

Brusatol showed dose dependent growth inhibitory effects with HL-60 (2.5×10⁶ cells/ml) cells. From 0.25 mg/kg to 5 mg/kg, brusatol inhibited the HL-60 cells at both i.p. and s.c. sites without causing significant weight loss, the inhibitory effect at i.p. site was ranging from 88.5% to 100%; and at s.c. site, the inhibitory percentage was around 25%, except when the compound dose went up to 5 mg/kg, 80.8% of inhibition was observed at s.c. site. At 12.5 mg/kg, brusatol was lethal to mice (FIG. 5).

The potential of brusatol and bruceantin to induce differentiation, antiproliferative, and differential cytotoxic effects in a panel of eleven leukemic cell lines has been demonstrated. Cell growth and differentiation studies with this panel revealed two patterns of activity. One group of cell lines, namely HL-60, K562, Kasumi-1, and Reh, was less responsive to brusatol or bruceantin mediated cytotoxicity, but their growth was arrested at the G₁ phase. Further, these cells (with the exception of Reh) demonstrated some degree of differentiation, based on one or more markers of this process. The second group, comprised of NB4, U937, BV173, SUPB13, RS4;11, Daudi, and DHL-6 cells, were extremely sensitive to brusatol or bruceantin, as shown by marked cytotoxic effects, but little induction of differentiation. Cell cycle analyses demonstrated apoptotic peaks with NB4 and BV173, an arrest in G₁ phase with SUPB13, and an arrest in S phase with U937 and RS4;11, suggesting different cytotoxic mechanisms may be triggered. Although the reason for the difference in the response of the various cell lines is unknown, it was observed that brusatol exerts strong cytotoxicity in those cell lines reported to express wild-type p53, including NB4, U937, BV173, and Daudi, while some of the less sensitive cell lines have been reported to be p53-null or mutant p53-expressing cell lines, e.g., HL-60, K562, Kasumi-1, and Reh.

The mechanism of action of various differentiation and apoptosis inducers remains largely unknown, but the participation of certain key genes have been demonstrated for some active compounds, such as ATRA and CGP 57148. Evaluation of c-Myc mRNA and protein expression in our panel of leukemic cell lines revealed brusatol and bruceantin induced marked decreases. However, with the exceptions of Kasumi-1, SUPB13, and Reh cells, down-regulation of c-Myc mRNA was less intense than the decrease observed with c-Myc protein levels. These data suggest translational (e.g., regulation of the internal ribosome entry segment of c-Myc mRNA) and/or post-translational (e.g., ubiquitination by proteasome complexes) regulation of this oncogene. Brusatol- and bruceantin-mediated early down-regulation of c-Myc correlated with induced differentiation in various cell lines, including monocytic differentiation in HL-60, K562, NB4, and U937, and moderate erythrocytic differentiation in BV173 and RS4;11. Cell death induction in NB4, U937, BV173, RS4;11, and Daudi cells also correlated with decreases of c-Myc, particularly at 24 hours.

The biological consequences of down-regulating c-Myc are numerous. In the hematopoietic system, this gene inhibits differentiation, and functions as a leukemogenic protein in various lymphomas and leukemias. Moreover, it is known that deregulation of c-Myc, in conjunction with p53 and bcl-2 mutations, is associated with malignant phenotype. For instance, chronic myelogenous leukemia cell lines possessing negligible levels of wild-type p53 (like K562) also expressed high levels of c-Myc, while the reverse phenomenon is observed in CML cell lines that express high levels of wild-type p53 (such as BV173). These and other studies have led to the hypothesis that myc deregulation decreases the probability of maturation, while p53 and bcl-2 mutations enhance cell survival, therefore favoring leukemic cell renewal. Thus, it is theorized, but not relied upon, that brusatol-induced c-Myc down-regulation could trigger cell death mechanisms preferentially in those cell lines with wild-type p53 protein expression, while triggering terminal differentiation in other cell lines with genetic defects in their apoptotic pathways.

In summary, it has been shown that quassinoids mediate strong cytotoxic effects in various cell lines while sparing normal human lymphocytes, and inhibit proliferation primarily by producing a G₀/G₁ arrest. This arrest is associated with subsequent expression of various markers of differentiation, and differentiation effects are irreversible following 48 hour drug exposures. In addition, cell lines that were most sensitive to brusatol-mediated cytotoxicity were eliminated with only 48 hours of exposure. Notably cytotoxic or differentiating effects were observed in the concentration range of 10 to 100 ng/ml, and 25 ng/ml was a sufficient in vitro concentration (10 ng/ml for bruceantin) to mediate these growth inhibitory responses. This is of importance since pharmacokinetic studies with human beings have demonstrated that a single intravenous injection of 3 mg/m² bruceantin can yield a blood level of 22 ng/ml. Moreover, this dose was well tolerated with few side effects, including a lack of hematologic toxicity, and normal lymphocytes were considerably less sensitive to the cytotoxic effects of brusatol or bruceantin. These observations suggest that a nontoxic concentration of brusatol administered for a short exposure time is sufficient to induce differentiation followed by cell death without the necessity of prolonged treatments. Biological responses correlate with potent down-regulation of c-Myc. Activity of these quassinoids has been demonstrated with the in vivo hollow fiber model with HL-60 cells, as discussed above. If similar mechanisms are found to apply in animal models of leukemia, a compelling argument would exist for evaluating clinical usefulness in leukemic patients.

In an effort to discover new chemotherapeutic/chemopreventive agents from natural sources, brusatol was found to induce HL-60 cellular differentiation, accompanied by strong antiproliferative and cytotoxic effects. A series of natural and semisynthetic quassinoids (identified hereafter as compounds 1-48) was designed to effect both anti-proliferative and differentiation inducing properties. Compounds were assessed in vitro using the HL-60 promyelocytic cell model. Changes in activity due to structural modification of the core structure of glaucarubolone (24) were consistent with activities reported in other cell systems. However, the following were novel SAR findings: (a) semisynthetic analogues with a hydroxylated ring at the b-position of the ester side chain at C-15 were able to induce cellular differentiation at concentrations lower than those inducing cell growth arrest, and (b) quassinoids inhibiting DNA synthesis with greater efficacy than reducing cellular viability possessed alkyl substitutions at the a-position of the C-15 ester side chain. Analogues from this latter group, and brusatol (1) and bruceantin (2), inhibited dimethylbenz(a)anthracene-induced preneoplastic lesion formation in a mouse mammary organ culture. The novel finding of brusatol and glaucarubolone analogues as potent inducers of differentiation leads to novel applications in the field of cancer.

The concept that aberrant cell differentiation is a consistent and important characteristic of malignant cells has been exploited to develop novel chemotherapeutic and/or chemopreventive agents. Evidence that induction of differentiation is sufficient to control malignancy was obtained from studies using somatic cell hybridization. It has been demonstrated that malignant cells fused with normal diploid cells of the same species result in hybrid cells that retain their transformed phenotype in culture. However, when inoculated into immune-deficient animals, these cells fail to form tumors due to induction of differentiation in the host animal. In a similar manner, nonphysiological agents are known to induce differentiation in malignant cells that have lost their normal response to the physiological inducers of maturation.

The HL-60 cell system has been utilized as a tool to study the molecular and cellular events that lead to maturation. Various chemical entities have shown remarkable activities as inducers of HL-60 cell differentiation. These compounds act through gene expression regulation of important signals that regulate differentiation, proliferation, and cell death processes. For instance, all-trans-retinoic acid was discovered as a differentiating agent using this system, and together with its natural and synthetic analogues, constitutes one of the most important categories of chemopreventive and chemotherapeutic agents.

In a search for novel anticancer agents, the HL-60 system was utilized as a screening tool of natural sources, and this led to the isolation of brusatol (1) from the seed extract of Brucea javanica (Simaroubaceae) as a potent natural inducer of cellular differentiation. Brusatol belongs to the chemical type of nortriterpenoids termed quassinoids (simaroubolides), which are biogenetically derived by degradation of C₃₀-precursors. These compounds are known to mediate several biological activities including antileukemic and cytotoxic responses. The major mechanism responsible for antineoplastic activity at the molecular level by the quassinoids has been attributed to inhibition of site-specific protein synthesis. Such inhibition has been shown to occur via interference at the peptidyltransferase site, thus preventing peptide bond formation. However, quassinoids are not universal protein synthesis inhibitors. They mediate cytotoxic effects with normal and transformed lymphocytic and hepatic cell lines, while enhancing proliferation of normal and transformed kidney and lung cells. Further, it has been demonstrated more complex mechanisms involving down-regulation of nm23 and c-Myc.

The potential of 48 quassinoids to induce HL-60 cell differentiation was evaluated and structure-activity relationships (SAR) determined was investigated. As an initial evaluation of the relevance of these effects, a group of selected quassinoids was tested for their potential to inhibit dimethylbenz(a)anthracene (DMBA)-induced preneoplastic lesions in a mouse mammary organ culture.

The following set of 48 natural and semi-synthetic quassinoid analogues (1-48) was studied using the HL-60 system to determine SAR. The numbering systems of quassinoids evaluated for potential to induce cell differentiation or inhibition of preneoplastic lesion formation in MMOC also is provided.

Brusatol Series

| | |
|---|---|
| 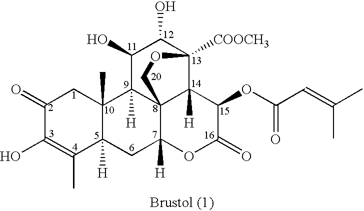 | 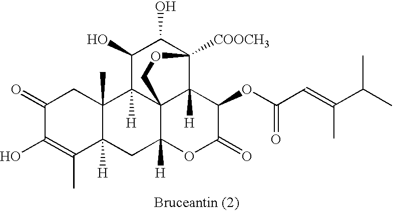 |
| 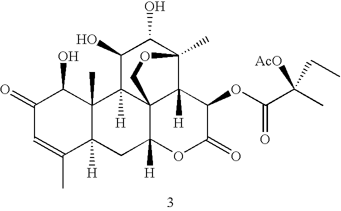 | 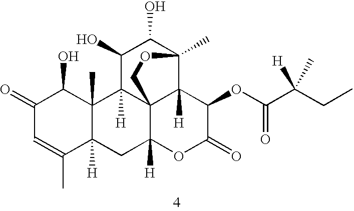 |
| 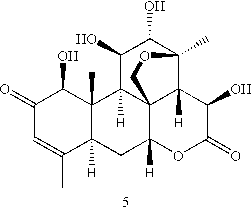 | 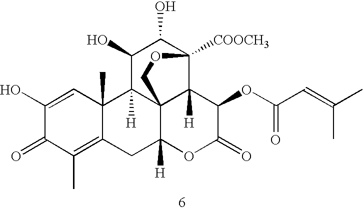 |
| 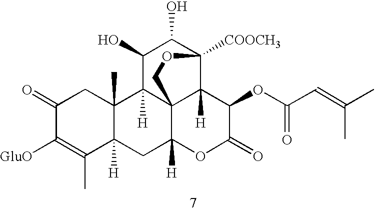 | |

Glaucarubolone Series

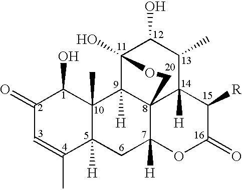

R =

| | | | | | |
|---|---|---|---|---|---|
| 8 | 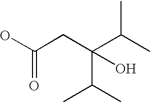 | 16 | 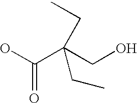 | 24 | OH (Glaucarubolone) |
| 9 | 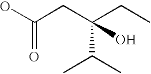 | 17 | 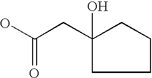 | 25 | 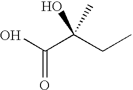 |
| 10 | 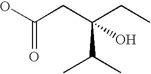 | 18 | 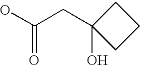 | 26 | H (Chaparrinone) |
| 11 | 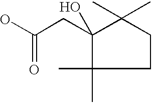 | 19 | 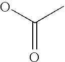 | 27 | 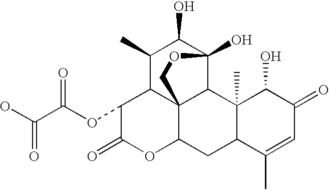 |
| 12 | 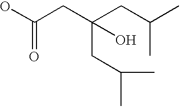 | 20 | 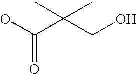 | 28 | 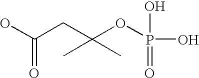 |
| 13 | 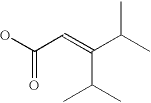 | 21 | 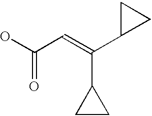 | 29 | 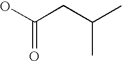 |
| 14 | 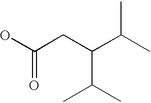 | 22 | 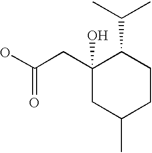 | 30 | 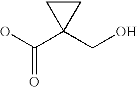 |
| 15 | 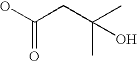 | 23 | 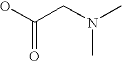 | 31 | 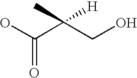 |
| | | | | 32 | 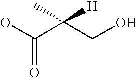 |
| | | | | 33 | 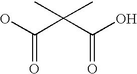 |
| | | | | 34 | 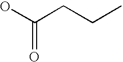 |
| | | | | 35 | 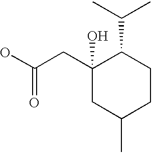 |

| | |
|---|---|
| 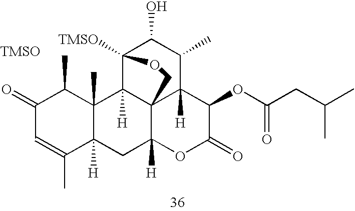 | 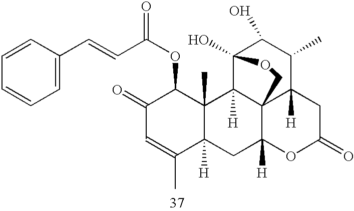 |
| 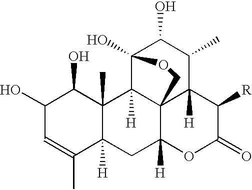 | 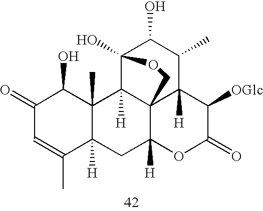 |
| 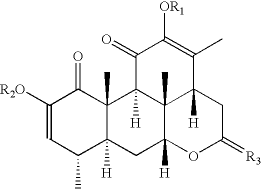 | 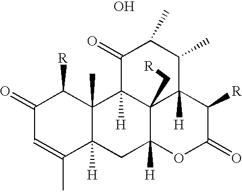 |
| 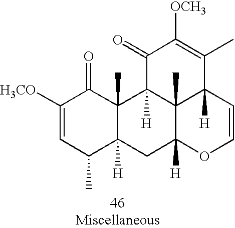 | 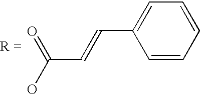 |
| 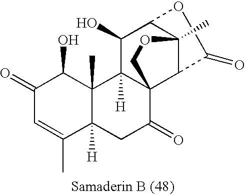 | 38 R = OH |
| Quassin Series | 39 R = OGlc |
| R₁ R₂ R₃ | 40 R = H |
| 43 CH₃ CH₃ ═O | 41 R = OCOCH₃ |
| 44 H H ═O | |
| 45 CH₃ CH₃ —H, —OH | |

Induction of differentiation was determined by the ability of treated cells to produce superoxide anions (nitroblue tetrazolium (NBT)-reduction), a functional marker of mature macrophages or granulocytes. Proliferation capacity is equivalent to cell growth and was measured by incorporation of [³H]thymidine into DNA over a period of 18 h, and cytotoxic activity was evaluated by the loss of membrane integrity as shown by trypan blue exclusion. Thirty-three quassinoids showed activity as either cytotoxic, antiproliferative, and/or inducers of cellular differentiation (Table 6). Inactive quassinoids (IC₅₀>5 mM) lacked either the epoxymethano-bridge in ring D (i.e., quassin series 43-47), or a free hydroxy group at positions 1, 3, 11, and 12 (i.e., due to glycosylation, compounds 7, 39, 42), or a freely conjugated ketone in ring A (i.e., 6, and due to reduction, compounds 38-41). Although members of the brusatol and glaucarubolone series were active, when comparing members of both series that varied only in the positioning of the epoxymethano bridge, great differences were noted, as shown with yadianzolide C (5) and glaucarubolone (24), and analogues 4 (brusatol series) and 32 (glaucarubolone series).

TABLE 6
Induction of HL-60 cell differentiation
and growth arrest by quassinoids (1-48)^{a}

| | Inhibition | Induction | | | Differentiation |
|---|---|---|---|---|---|
| | of Prolif- | of Differen- | | | on Selectivity* |
| | eration | tiation | Cytotoxicity | Selectivity | Index |
| Compound | (IC₅₀, μM) | (EC₅₀, μM) | (IC₅₀, μM) | Index^{b} | Cytotox/diff |
| 1 | 0.07 ± 0.007 | 0.07 ± 0.001 | 0.17 ± 0.001 | 2.5 ± 0.3 | 2.5 |
| 2 | 0.04 ± 0.003 | 0.02 ± 0.002 | 0.04 ± 0.003 | 1.0 ± 0.2 | 2.0 |
| 3 | 0.06 ± 0.005 | 0.09 ± 0.001 | 0.13 ± 0.008 | 2.2 ± 0.5 | 1.4 |
| 4 | >0.2 | >0.2 | 0.2 ± 0.004 | — | — |
| 5 | 1.5 ± 0.08 | 10 ± 1.5 | >10 | >6.7 | 1.0 |
| 8 | 0.009 ± 0.0007 | 0.009 ± 0.0002 | 0.009 ± 0.0007 | 1.0 ± 0.2 | 1.0 |
| 9 | 0.011 ± 0.0004 | 0.017 ± 0.0004 | 0.013 ± 0.001 | 1.2 ± 0.2 | 0.8 |
| 10 | 0.009 ± 0.001 | 0.019 ± 0.0004 | 0.013 ± 0.001 | 1.5 ± 0.4 | 0.7 |
| 11 | 0.04 ± 0.008 | 0.025 ± 0.002 | 0.05 ± 0.004 | 1.3 ± 0.5 | 2.0 |
| 12 | 0.04 ± 0.003 | >0.05 | 0.04 ± 0.002 | 1.0 ± 0.2 | <0.8 |
| 13 | 0.07 ± 0.003 | 0.09 ± 0.003 | 0.13 ± 0.01 | 1.9 ± 0.3 | 1.4 |
| 14 | 0.075 ± 0.009 | 0.055 ± 0.01 | 0.15 ± 0.02 | 2.0 ± 0.7 | 2.7 |
| 15 | 0.1 ± 0.02 | ˜0.2 ± 0.004 | 0.4 ± 0.02 | 4.2 ± 1.5 | −2.0 |
| 16 | 0.15 ± 0.02 | 0.5 ± 0.05 | 0.95 ± 0.02 | 6.5 ± 1.4 | 1.9 |
| 17 | 0.19 ± 0.04 | 0.13 ± 0.01 | 0.38 ± 0.04 | 2.1 ± 0.9 | 2.9 |
| 18 | 0.26 ± 0.009 | 0.14 ± 0.003 | 0 28 ± 0.01 | 1.1 ± 0.1 | 2 0 |
| 19 | 0.2 ± 0.01 | 0.2 ± 0.001 | 0.5 ± 0.07 | 2.5 ± 0.7 | 2.5 |
| 20 | 0.2 ± 0.02 | 0 5 ± 0.01 | 0.6 ± 0.08 | 3.1 ± 1 | 1.2 |
| 21 | 0.2 ± 0.03 | 0.6 ± 0.07 | 0.75 ± 0.02 | 3.9 ± 1 | 1.3 |
| 22 | 0.4 ± 0.03 | 0.3 ± 0.008 | 0.9 ± 0.1 | 2.3 ± 0.6 | 3.0 |
| 23 | 0.5 ± 0.03 | 0.5 ± 0.04 | 0.8 ± 0.1 | 1.6 ± 0.4 | 1.6 |
| 24 | 0.4 ± 0.03 | ˜1.3 ± 0.03 | 1.3 ± 0.2 | 3.3 ± 1 | ˜1.0 |
| 25 | 0.5 ± 0.09 | 1.8 ± 0.09 | 1.0 ± 0.1 | 2.1 ± 0.8 | 0.6 |
| 26 | 1.2 ± 0.2 | ˜2.4 ± 0.1 | 5.0 ± 0.3 | 4.3 ± 1.4 | ˜2.1 |
| 27 | 0.5 ± 0.04 | >1.5 | 1.8 ± 0.1 | 3.6 ± 0.7 | <1.2 |
| 28 | 0.7 ± 0.09 | 3.0 ± 0.03 | 4.4 ± 0.3 | 6.5 ± 1.7 | ˜1.5 |
| 29 | 1.0 ± 0.1 | >2.0 | 1.5 ± 0 1 | 1.5 ± 0.4 | <0.7 |
| 30 | 1.0 ± 0.15 | 2.0 ± 0.03 | 2.8 ± 0.2 | 2.9 ± 0.9 | ˜1.4 |
| 31 | 0.6 ± 0.1 | 1.9 ± 0.04 | 2.5 ± 0.05 | 4.3 ± 1.1 | 1.3 |
| 32 | 1.5 ± 0.2 | >6.0 | 6.2 ± 0.06 | 4.2 ± 0.9 | <1.0 |
| 33 | 3.2 ± 0.35 | >7.0 | >7.0 | >2.2 | ˜1.0 |
| 34 | >3.0 | >3.0 | 3.0± | — | <1.0 |
| 48 | 0.4 ± 0.05 | 1.0 ± 0.02 | >2 | >6 | >2.0 |

^{a}[³H]Thydimidine incorporation was used as a proliferation marker, NBT-reduction as a differentiation marker, and trypan blue exclusion as a viability marker. Inhibitory (IC₅₀) and effective (EC₅₀) concentrations required to induce a 50% response were determined using dose-response studies with at least five different data points. Compounds 6, 7, 35-47 were tested and found to be inactive (IC₅₀ > 5 μM).
^{b}Selectivity index was calculated as the ratio of cytotoxic IC₅₀ over antiproliferative IC₅₀.
*Differentiation Selectivity Index was calculated as the ratio of the cytotoxic IC₅₀ over differentiation EC₅₀.

The effect of an ester side chain at position C-15 on cytotoxicity and cellular differentiation was studied in greater detail with analogues of glaucarubolone (compounds 8-35). The absence of a side,chain at C-15 is associated with a 100-500-fold decrease of potency (compared to brusatol (1)), and an increased selective inhibition of DNA synthesis as compared to cytotoxicity (i.e., compounds 5, 24 and 26).

The nature of the side chain also is important. There were no correlations between the lipophilicity of the ester side chain and HL-60 cell differentiation induction. Analysis of two pairs of enantiomers (9-10 and 31-32) revealed that the stereochemistry at the α- or β-positions does not affect biological activity. On the contrary, the addition of alkyl groups resulting in a branched side chain correlated with increase in potency as shown between side chains 11 and 17, 14 and 29, and 16 and 20. The presence of hydroxyl substituents at the β-position of the side chain correlated with 8-10-fold increase in potency as shown by comparing the following pairs: 8 with 14, and 15 with 29. The presence of alkyl substituents in the α-position correlated with increased selectivity (2-6-fold) for antiproliferative activity versus cytotoxicity, but less potency as inducers of differentiation (analogues 16, 20, 25, 30-33).

An increase in selectivity (1.5-2-fold) between induction of differentiation and anti-proliferative/cytotoxic activity was observed only in those analogues possessing side chains with cyclic rings in the β-position (11, 17, 18 and 22). Others were cytotoxic but not antiproliferative or inducers of cellular differentiation (4, 34). In sum, novel esters of glaucarubolone (24) were shown to be either more potent or more selective than the parent compound and brusatol (1).

A smaller set of quassinoids (i.e., compounds 1, 2, 10, 14, 16, 18, 26, 34, and 48) was tested for potential to inhibit DMBA-induced preneoplastic lesion formation in the mouse mammary organ culture (MMOC) model. This model correlates with in vivo chemopreventive activity in models such as the DMBA-induced rat mammary adenocarcinoma and the DMBA/12-O-tetradecanoylphorbol 13-acetate (TPA) two-stage mouse skin papilloma models. All nine quassinoids were tested at the same concentration (2 mM). Four were active (Table 7). Interestingly, potency in the HL-60 assay did not correlate with activity in the MMOC assay, considering that quassinoids 10 and 14 were among the most potent inducers of HL-60 differentiation (EC50<0.05 mM), but inactive in the MMOC model at concentrations as high as 2 mM. Activity in the MMOC assay seemed to favor analogues having alkyl substituents at the α-position of the C-15 ester side chain (Table 7), contrary to that observed in HL-60 where potency correlated with the presence of β-branched ester side chains (Table 7). For instance, compound 34 was only moderately antiproliferative in the HL-60 cell line (EC₅₀=3.2 mM), but greatly inhibited preneoplastic lesion formation (82% inhibition).

TABLE 7
Quassinoid-inhibition of DMBA-induced
Preneoplastic Lesion Formation Using the Mouse
Mammary Organ Culture Model

| Quassinoid (2 μM) | % inhibition^{a} |
|---|---|
| brusatol (1) | 70 |
| bruceantin (2) | 70 |
| 10 | 44 |
| 14 | 40 |
| 16 | 70 |
| 18 | 33 |
| chaparrinone (26) | 25 |
| 34 | 82 |
| samaderin B (48) | 0 |

^{a}Percent inhibition was calculated in comparison with a DMBA (carcinogen) contro1. Based on historical controls26, samples are classified as active if preneoplastic lesions are reduced to >60%. No visible signs of toxicity (as indicated by dilation of mammary ducts or disintegration of mammary structure yielding amorphous material) was manifested by any of the tested quassinoids.

It has been reported that quassinoids regulate DNA and RNA synthesis by blocking several metabolic sites necessary for nucleic acid synthesis, while protein synthesis is regulated by binding to the ribosome. Inhibition of protein synthesis has been linked to cytotoxicity and anti-neoplastic activity of quassinoids, since resistant tumors and cell lines are still sensitive to quassinoid-inhibition of DNA and RNA synthesis while resistant to protein synthesis inhibition. In the current study, utilizing HL-60 cells in culture, quassinoids were antiproliferative agents and potent inducers of cellular differentiation.

As illustrated through analysis of 48 quassinoids using the HL-60 cell system, inhibition of DNA synthesis/cellular growth and potential to induce differentiation are greatly influenced by structural alterations. As demonstrated by previous literature reports, some correlations can be drawn with antineoplastic activity, but exceptions are obvious. For example, brusatol dimers are more potent as antineoplastic agents than brusatol (1) itself, whereas brusatol is more potent as an anti-inflammatory or differentiating agent. Analogues that lack an ester side chain inhibitor DNA synthesis at lower concentrations than those required to inhibit cellular growth (and protein synthesis), while other analogues were cytotoxic without inhibiting DNA synthesis. These data suggest that selectivity for a particular cellular target can be achieved by structural modification of the parent quassinoid.

Extensive studies on agents that induce metabolic arrest show a correlation between DNA synthesis inhibitors and induction of differentiation. For instance, the inhibition of DNA synthesis has been shown to be an initial event necessary to induce cell differentiation by antineoplastic agents such as ara-C and actinomycin D. It has been proposed that inhibition of DNA synthesis allows the slow production of some proteins necessary for fulfillment of the differentiation program. However, SAR studies demonstrated that some quassinoids with potent antiproliferative activity did not induce differentiation, and analogues 4 and 33 were cytotoxic, but neither antiproliferative nor differentiation inducers. In addition, known inhibitors of DNA synthesis, i.e., aphidicolin, were incapable of inducing maturation of HL-60 cells (data not shown). These observations make it unlikely that inhibition of DNA synthesis is the mechanism of induction of differentiation.

Certain protein synthesis inhibitors have also been reported to induce HL-60 cell differentiation. Although inhibition of protein synthesis and gene expression activation seem to be mutually exclusive events, some reports have shown that selective gene expression and translation can occur with as little as 10% of control protein synthesis levels. Several theories have been proposed for the observed results. One is that inhibitors that act by blocking the elongation step of protein synthesis, like the quassinoids, increase the stability of weak mRNAs and decrease the degradation of certain proteins necessary for the induction of differentiation. In support of this idea, quassinoids and Cephalotaxus alkaloids (i.e., homoharringtonine) are efficient differentiating agents that bind to similar sites in the ribosome. Quassinoids and Cephalotaxus alkaloids induce disaggregation of polyribosomes, while other protein synthesis inhibitors (cycloheximide and anisomycin) function by other modes of action and are not capable of inducing cellular differentiation. Studies on differentiation of cell lines with mutated ribosomal sites would clarify this issue.

The differentiation-inducing and anti-proliferative effects of retinoic acid was identified first with the HL-60 cell line, and confirmed with other cell systems. Subsequently, studies with in vitro and in vivo chemically induced models of carcinogenesis established a correlation between induction of differentiation and chemopreventive activity, e.g., inducers of cell differentiation inhibit preneoplastic lesion formation in MMOC26 and adenocarcinomas in the Sprague-Dawley rat mammary model. Moreover, retinoic acid and novel retinoids have shown chemopreventive activity against primary and secondary tumor formation in human clinical trials of lung and head and neck cancers. In the present study, initial assessment of the chemopreventive potential of brusatol (1) and glaucarubolone esters was performed using the MMOC model. It was found that analogues bearing a-dialkylated C-15 ester side chains were more selective in the cell differentiation tests, as well as being active in the MMOC model.

### EXPERIMENTAL SECTION

### Preparation of Quassinoids

Brusatol (1), yadanziolide C (5), dehydrobrusatol (6) and bruceoside A (7) were isolated from Brucea javanica, and bruceantin (2) was obtained from the NCI. Quassinoids belonging to the glaucarubolone series (37-42, 47) and quassin series (43-46) were obtained by J. D. and J. D. M. Peninsularinone (10) was isolated from Castela peninsularis. Glaucarubolone (24), glaucarubinone (25), chaparrinone (26), samaderin B (48), quassimarin (3), and simalikalactone D (4), were prepared via total synthesis. Semisynthetic analogues 8, 9, 11-23, and 27-36 were prepared via a four-step protocol starting with glaucarubolone (24), which was isolated from Castela polyandra.

### Differentiation/Proliferation and Cytotoxicity Assays using HL-60 Cells

HL-60 (human promyelocytic) cells were tested using a 4-day incubation protocol. In brief, cells in log phase (approximately 10⁶ cells/mL) were diluted to 105 cells/mL and preincubated overnight (18 h) in 24-well plates to allow cell-growth recovery. Then, samples dissolved in DMSO were added, keeping the final DMSO concentration at 0.1% (v/v). Control cultures were treated with the same concentration of DMSO. After 4 days of incubation, the cells were analyzed to determine the percentage of cells undergoing maturation as determined by NBT reduction. Concomitantly, the effect on viability and proliferation of HL-60 cells was determined. In each experiment, 1α,25-dihydroxyvitamin D₃ (EC₅₀=0.01 μM) and brusatol (EC₅₀=0.07 μM) were used as reference controls. EC₅₀ and IC₅₀ values were calculated using 5-7 test concentrations (in duplicate), and consistent results were obtained, indicating the data reported for the related quassinoids are reliable.

(1) Nitroblue Tetrazolium (NBT) Reduction. Evaluation of NBT reduction was used to assess the ability of sample-treated HL-60 cells to produce superoxide when challenged with 12-O-tetra-decanoylphorbol 13-acetate (TPA). A 1.1 (v/v) mixture of a cell suspension (10⁶ cells) and TPA/NBT solution (2 mg/mL NBT and 1 μg/mL TPA in phosphate buffered solution) was incubated for 1 h at 37° C. Positive cells reduce NBT yielding intracellular black-blue formazan deposits, which were quantified by microscopic examination of >200 cells. Results were expressed as a percentage of positive cells.

(2) Cytotoxicity. Since loss of membrane integrity is an early feature of necrotic cells and a late feature of apoptotic cells, trypan blue, a cationic blue dye, was used to stain cells with compromised plasma membranes, while leaving intact cells unstained. Cells (100 mL) were stained with 400 mL of trypan blue (0.2% w/v in PBS), incubated for at least 5 min at room temperature, and counted using a hematocytometer. Viability percentages were calculated with duplicate samples.

(3) Cell Proliferation Assay. Inhibition of [³H]thymidine incorporation into DNA was determined to assess the level of HL-60 cell proliferation. Cells were treated with the test samples for four days, then placed into 96-well plates (100 μL) and treated with [³H]thymidine (0.5 μCi/ml, 65 Ci/mmol) for 18 h at 37° C. in a 5% CO₂ incubator. Cells were then collected on glass fiber filters (90×120 mm; Wallac, Turku, Finland) using a TOMTEC Harvester 96®. The filters were counted using a Microbeta™ liquid scintillation counter (Wallac, Turku, Finland) with scintillation fluid. Finally, the percentage of [³H]thymidine incorporation per 10⁶ cells was calculated by dividing the dpm of sample-treated cells by the dpm of DMSO-treated cells.

Inhibition of DMBA-Induced Preneoplastic Lesion Formation in Mouse Mammary Organ Culture (MMOC)

The identification of potential inhibitors of DMBA-induced preneoplastic lesion formation in mammary organ culture has been described previously. Briefly, four-week old BALB/c female mice (Charles River) were pretreated for nine days with 1 μg estradiol and 1 mg progesterone. The thoracic pair of mammary glands was dissected on silk and incubated with growth-promoting hormones in the presence of test compounds (2 μM) for 10 days. DMBA (2 mg/mL) was included in the medium (containing 5 mg/mL insulin, 5 mg/mL prolactin, 1 mg/mL aldosterone, and 1 mg/mL hydrocortisone) for 24 hours on the third day of culture to induce preneoplastic mammary lesions. Following 10 days of growth promoting phase, all hormones except insulin were withdrawn and the glands were allowed to regress to lobuloalveolar structures during a 14-day incubation period. Glands then were fixed in 10% buffered formalin and stained with alum carmine. Incidence of lesion formation (percentage of glands per group with mammary lesions) was recorded, and percent inhibition was calculated by comparison with the DMBA control group that was not treated with test sample. Active samples induce 60% inhibition, based on historical controls.

Additional studies directed to the anti-tumor activity of bruceantin focused on multiple myeloma using the RPMI 8226 cell line as a model. As discussed above, an important mechanism mediated by bruceantin is down-regulation of c-Myc. As shown in detail hereafter, with RPMI 8226 cells, c-Myc is strongly down-regulated by treatment with bruceantin. In addition, the cells undergo apoptosis, with an IC₅₀ value of approximately 7 ng/ml.

In vivo antitumor studies also were performed with male and female SCID mice. Six-week-old female SCID mice were inoculated with RPMI 8226 cells, and treated with various doses of bruceantin once a discernable tumor mass was present. At lower doses of bruceantin, tumor growth was completely inhibited without toxicity. At higher doses of bruceantin, loss of body weight or mouse lethality was observed. Using the same animals, treatment with bruceantin was initiated with the control group starting at 35 days postinoculation. Significant tumor growth inhibition was observed.

A similar experiment was performed with six-week-old male SCID mice. Bruceantin effectively inhibited the growth of RPMI 8226 cells in this experiment, similar to the effect observed with female SCID mice.

Effect of Bruceantin on c-Myc Expression Determined with RPMI 8226 Cell Line

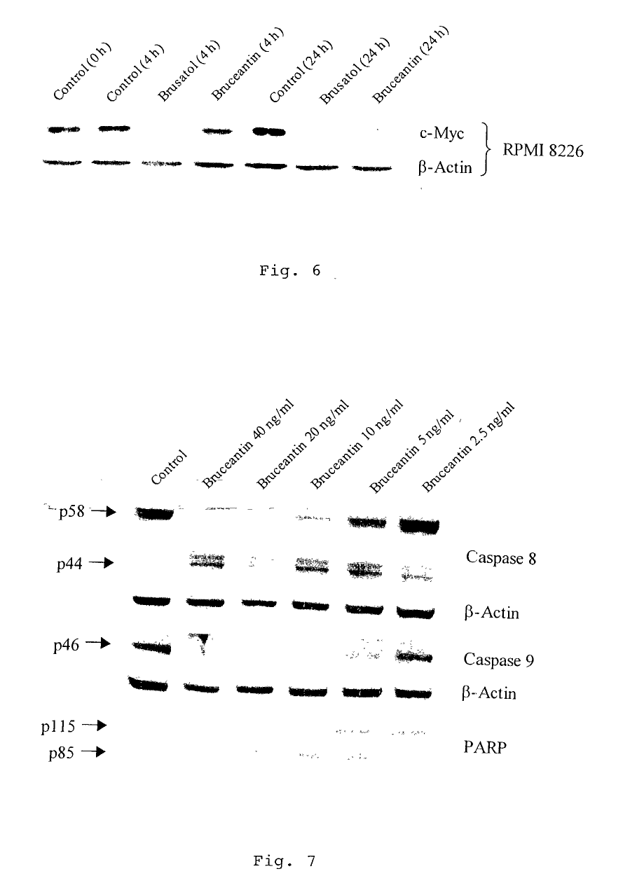

The expression of c-Myc protein was assessed by immunoblots. See FIG. 6. In brief, cells (10⁶) were treated with brusatol (25 ng/ml) or bruceantin (10 ng/ml) and harvested after 4 or 24 hours. Whole-cell pellets were lysed with detergent lysis buffer (1 ml/10⁷ cells, 50 mM Tris-HCl buffer, pH 8.0, 150 mM NaCl, 1 mM DTT, 0.5 mM EDTA, 1% Nonidet® P40, 0.5% sodium deoxycholate, 0.1% sodium dodecylsulfate, 100 μg/ml phenylmethylsulfonyl fluoride, 1 μg/ml aprotinin, 2 μg/ml leupeptin and 100 μM sodium vanadate) to obtain protein lysates, and protein concentrations were quantified using a bicinchoninic acid kit. Because c-Myc is labile, cell lysates were not frozen, but stored at 4° C. until all lysates were ready for a particular cell line, then the Western blot was performed. Total protein (30 μg) was separated by 10% SDS-PAGE, electroblotted to PVDF membranes, and blocked over night with 5% nonfat dry milk. The membrane was incubated with a 2.5 μg/ml solution of the primary antibody, prepared in 1% blocking solution, for 2 hours at room temperature, washed three times for 15 minutes with PBS-T (PBS with 0.1%, v/v, Tween 20), and incubated with a 1:2500 dilution oF horseradish peroxidase-conjugated secondary antibody for 30 minutes at 37° C. Blots again were washed three-times for 10 min each in PBS-T, and developed by enhanced chemiluminescence (Amersham). Membranes were exposed to Kodak Biomax film and the resulting film analyzed using Kodak 1D Image Analysis Software. Membranes then were stripped and reprobed for β-actin.

Effect of Bruceantin on Caspase-8, Caspase-9, and PARP Expression Determined with RPMI 8226 Cell Line

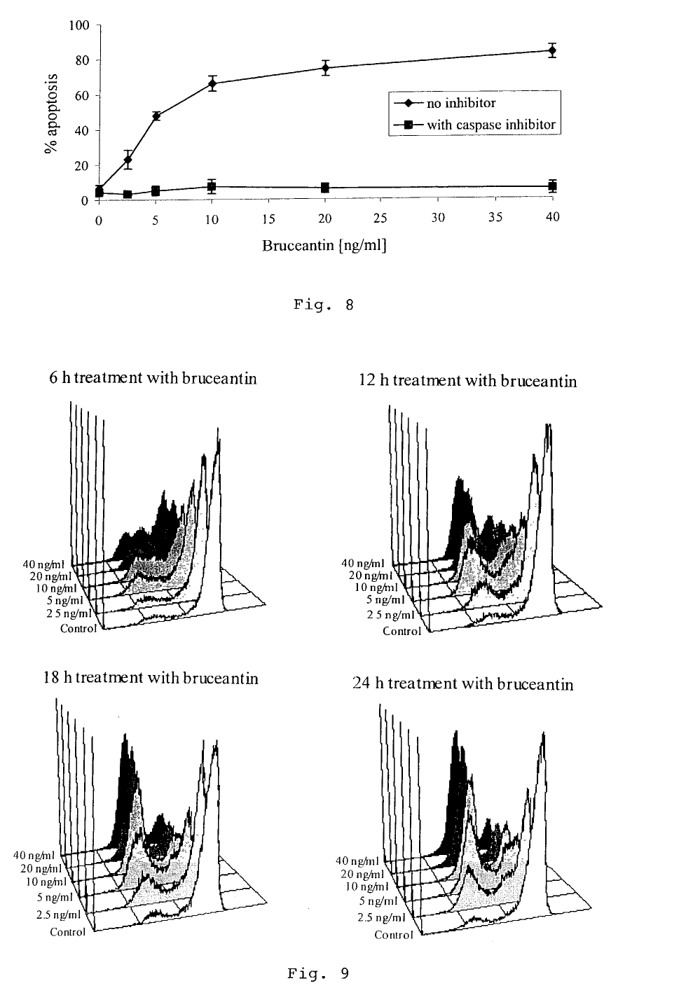

The expression of caspase-8, caspase-9, and PARP protein was assessed by immunoblots. See FIG. 7. In brief, cells (10⁶) were treated with bruceantin (2.5-40 ng/ml) and harvested after 24 hours. Whole-cell pellets were lysed with detergent lysis buffer (1 ml/10⁷ cells, 62.5 mM Tris-HCl buffer, pH 6.8, 6 M urea, 10% glycerol, 2% SDS, 0.00125% bromophenol blue and 5% β-mercaptoethanol), then sonicated for 15 seconds and incubated at 65° C. for 15 minutes to obtain protein lysates, and protein concentrations were quantified using a bicinchoninic acid kit. Total protein (30 μg) was separated by 7.5% or 10% SDS-PAGE, electroblotted to PVDF membranes, and blocked over night with 5% nonfat dry milk. The membrane was incubated with a 1:100-1:200 dilution of the primary antibody, prepared in 1% blocking solution, for 2 hours at room temperature, washed three times for 15 minutes with PBS-T (PBS with 0.1%, v/v, Tween 20), and incubated with a 1:2500 dilution of horseradish peroxidase-conjugated secondary antibody for 30 min at 37° C. Blots again were washed three times for 10 minutes each in PBS-T, and developed by enhanced chemilum-inescence (Amersham). Membranes were exposed to Kodak Biomax film, and the resulting film analyzed using Kodak 1D Image Analysis Software. Membranes then were stripped and reprobed for β-actin.

DAPI staining

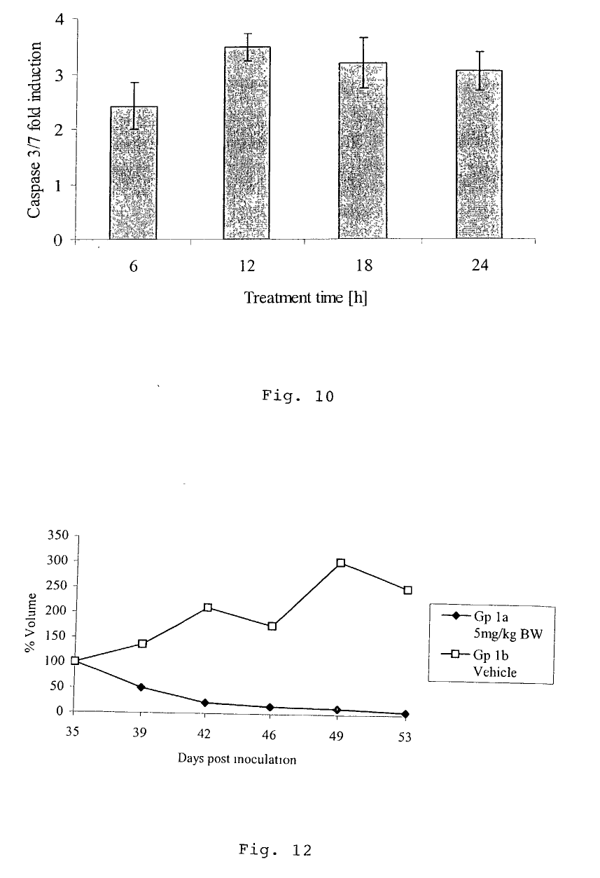

Cells were treated with different concentrations of bruceantin (2.5, 5, 10, 20, or 40 ng/ml) for 24 hours, washed with PBS, and fixed with methanol:acetic acid 1:1 for 30 minutes at room temperature. Cells then were treated with DAPI (1 μg/ml) for 15 minutes at room temperature. DAPI staining of the cells was observed by fluorescence microscopy. At least 100 cells were counted for each sample. Dose-response curves showing the percent apoptosis with or without caspase inhibitor were constructed (FIG. 8), and the concentration of bruceantin required to induce apbptosis in 50% of the cell population (IC₅₀) was calculated as 9.2 ng/ml. Etoposide (10 μM) was used as a positive control. The same experiment was performed, adding a caspase 1, 3, 4, and 7 inhibitor one hour prior treatment with bruceantin.

Effect of Bruceantin on Mitochondrial Membrane Potential in RPMI 8226 Cell Line

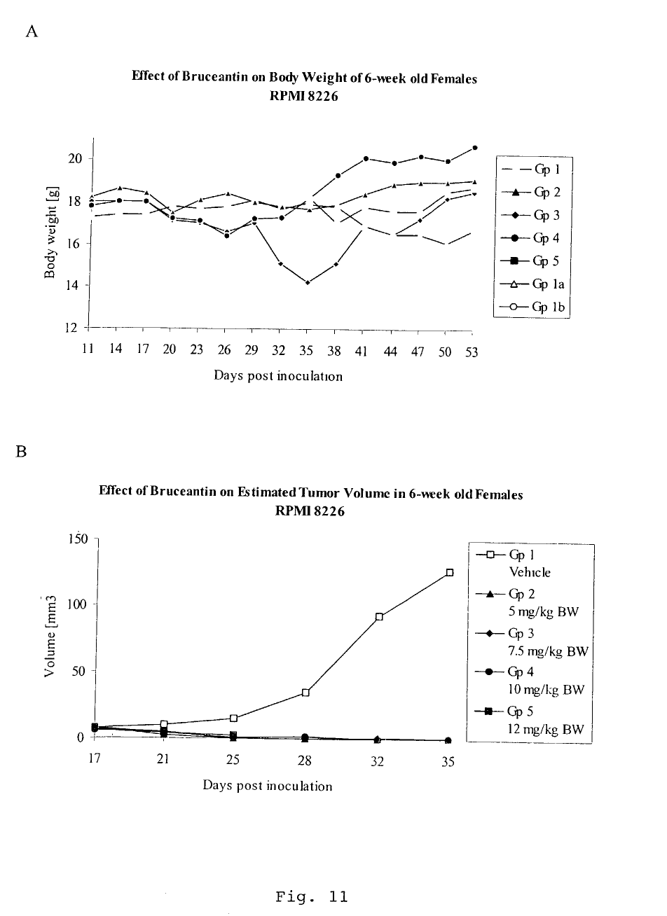

3′,3′-Dihexyloxacarbocyanine iodide (DiOC₆) is a dye used to measure mitochondrial membrane potential. In brief, cells (5×10⁶) were treated with different concentrations of-bruceantin (2.5, 5, 10, 20, or 40 ng/ml) for 6, 12, 18, or 24 hours. Fifteen minutes prior to collection of cells after drug treatment, DiOC₆ (40 nM) was added to the cells. Cells were washed once with PBS before resuspending in 300 μl PBS containing 40 nM DiOC₆ and 30 μg/ml propidium iodide (PI). Fluorescence intensities of DiOC₆ were analyzed by flow cytometry with excitation and emission settings of 484 and 500 nm, respectively. PI was added to gate out dead cells. Histograms (FIG. 9) show all PI negative cells.

Caspase-3 and -7 Activation by Bruceantin.

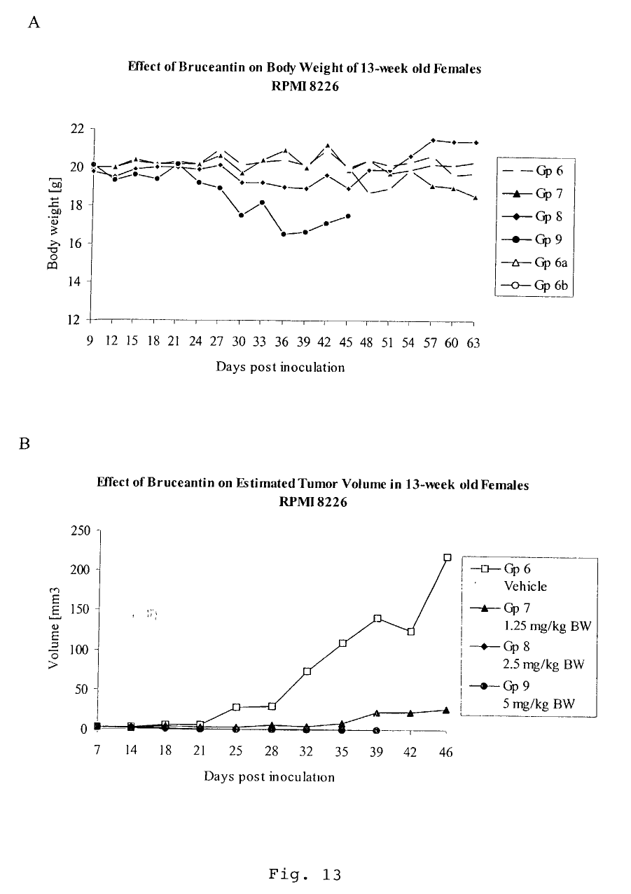

The Apo-ONE™ Homogeneous Caspase-3/7 Assay kit (Promega) was used to measure the activities of caspase-3 and -7. Cells (1.5×10⁴) were treated with bruceantin (10 ng/ml) for 0-24 hours in a black 96-well plate. At the end of the treatment, the buffer, which rapidly lyses/permeates the cells, and the substrate (rhodamine 110) were mixed, then were added to the cells. Upon sequential cleavage and removal of the DEVD peptides by caspase-3/7 activity and excitation at 499 nm, the rhodamine 110 leaving group becomes intensely fluorescent. The emission maximum is 521 nm. The amount of fluorescent product generated is proportional to the amount of caspase-3/7 cleavage activity present in the sample. The samples were measured in triplicate. Results are expressed in fold of induction compared to the control (i.e., DMSO-treated cells). See FIG. 10. The addition of a caspase inhibitor completely inhibited the signal amplification.

Protocols for Assessing Bruceantin with in vivo Antitumor Studies

Animals: female and male SCID mice at 6 or 13 weeks of age. Animals were quarantined for one week. The area of inoculation was shaved prior to inoculation. Animals were weighed twice weekly and observed daily.

Cell Line: 1×10⁷ RPMI 8226 cells were injected subcutaneously into the right rear flank of each animal. Cells were injected in a final volume of 0.1 ml.

Treatment: about 10-14 days post inoculation, or when tumor size was 5 mm, bruceantin (0-12 mg/kg body weight) was injected i.p. at three day intervals until the end of the study. Tumors were measured twice a week.

Bruceantin: bruceantin was dissolved in 100% ethanol, sonicated and diluted to a 5% ethanol solution with saline.

Duration: 50 days postinoculation.

TABLE 7
Protocols for assessing bruceantin
with in vivo antitumor studies

| Group | Cell line | Sex | Age (weeks) | Concentration (mg/kg BW) |
|---|---|---|---|---|
| 1 | RPMI 8226 | F | 6 | — |
| 2 | RPMI 8226 | F | 6 | 5 |
| 3 | RPMI 8226 | F | 6 | 7.5 |
| 4 | RPMI 8226 | F | 6 | 10 |
| 5 | RPMI 8226 | F | 6 | 12 |
| 6 | RPMI 8226 | F | 13 | — |
| 7 | RPMI 8226 | F | 13 | 1.25 |
| 8 | RPMI 8226 | F | 13 | 2.5 |
| 9 | RPMI 8226 | F | 13 | 5 |
| 10 | RPMI 8226 | M | 6 | — |
| 11 | RPMI 8226 | M | 6 | 2.5 |
| 12 | RPMI 8226 | M | 6 | 5 |
| 13 | RPMI 8226 | M | 6 | 7.5 |
| 14 | RPMI 8226 | M | 6 | 10 |

Effect of bruceantin on estimated tumor volume in six-week-old female SCID mice bearing established RPMI 8226 tumors

TABLE 8
Six-week-old female mice inoculated
with RPMI 8226 cells

| | | | |
|---|---|---|---|
| Days post | | | Tumor |
| inoculation | BW | Survival | Volume |

Group 1 (vehicle)

| | | | |
|---|---|---|---|
| 14 | 17.4 | 7 | NA |
| 17 | 17.4 | 7 | 7.7 |
| 20 | 17.8 | 7 | 9.8 |
| 23 | 17.7 | 7 | |
| 26 | 17.8 | 7 | 14.7 |
| 29 | 18.1 | 7 | 34.9 |
| 32 | 17.7 | 7 | 92.5 |
| 35 | 18.0 | 7 | 126.8 |

Group 2 (mg/kg EW)

| | | | |
|---|---|---|---|
| 14 | 18.6 | 7 | NA |
| 17 | 18.4 | 7 | 7.3 |
| 20 | 17.5 | 7 | 2.4 |
| 23 | 18.1 | 7 | |
| 26 | 18.4 | 7 | 0 |
| 29 | 18.0 | 7 | 0 |
| 32 | 17.8 | 7 | 0 |
| 35 | 17.7 | 6 | 0 |

Group 3 (7.5 mg/kg EW)

| | | | |
|---|---|---|---|
| 14 | 18.0 | 7 | NA |
| 17 | 18.0 | 7 | 6.7 |
| 20 | 17.1 | 7 | 5.3 |
| 23 | 17.0 | 7 | |
| 26 | 16.6 | 7 | 0 |
| 29 | 17.0 | 7 | 0 |
| 32 | 15.1 | 5 | 0.5 |
| 35 | 14.2 | 1 | 0 |

Group 4 (10 mg/kg BW)

| | | | |
|---|---|---|---|
| 14 | 18.0 | 8 | NA |
| 17 | 18.0 | 8 | 5.9 |
| 20 | 17.2 | 7 | 3.9 |
| 23 | 17.1 | 7 | |
| 26 | 16.4 | 5 | 0.8 |
| 29 | 17.2 | 4 | 1.3 |
| 32 | 17.3 | 1 | 0 |
| 35 | 18.2 | 1 | 0 |

Group 5 (12 mg/kg BW)

| | | | |
|---|---|---|---|
| 14 | 17.2 | 8 | NA |
| 17 | 17.5 | 8 | 7.2 |
| 20 | 16.7 | 7 | 4.1 |
| 23 | 16.2 | 6 | 2.8 |

| | |
|---|---|
| 26 | All mice are dead |
| 29 | |
| 32 | |
| 35 | |

Day 0 - Inoculation of RPMI 8226
Day 17 - First bruceantin inoculation
Day 32 - Last bruceantin injection for Groups 2-5
Day 35 - First bruceantin injection for Group 1a and no treatment for Groups 2-5.

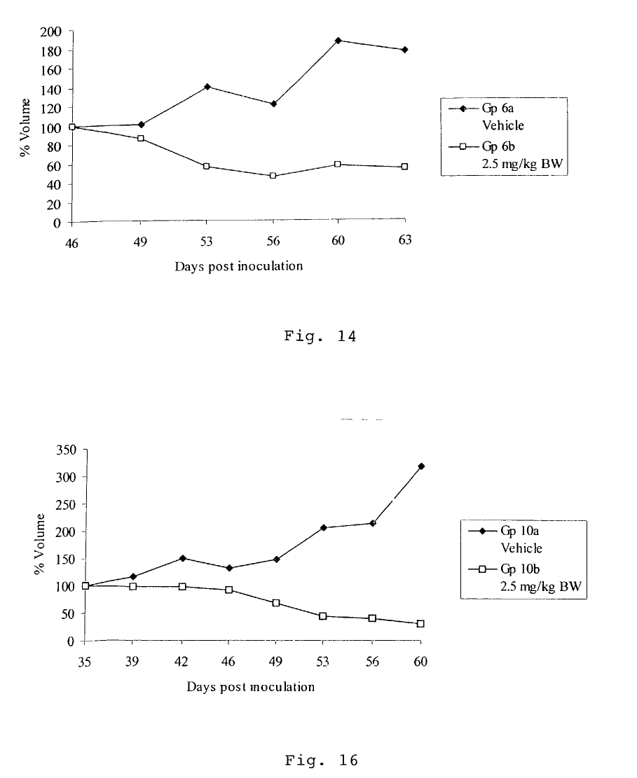

Thirty-two days post inoculation, the mice from Groups 2-5 received a final bruceantin treatment, and the mice from Group 1 were split into two groups: Group la containing five mice and Group lb containing two mice (see FIG. 11). Thirty-five days post inoculation, mice in Group la were administered bruceantin (5 mg/kg BW) every 3 days, and mice from Group 1b were administered vehicle.

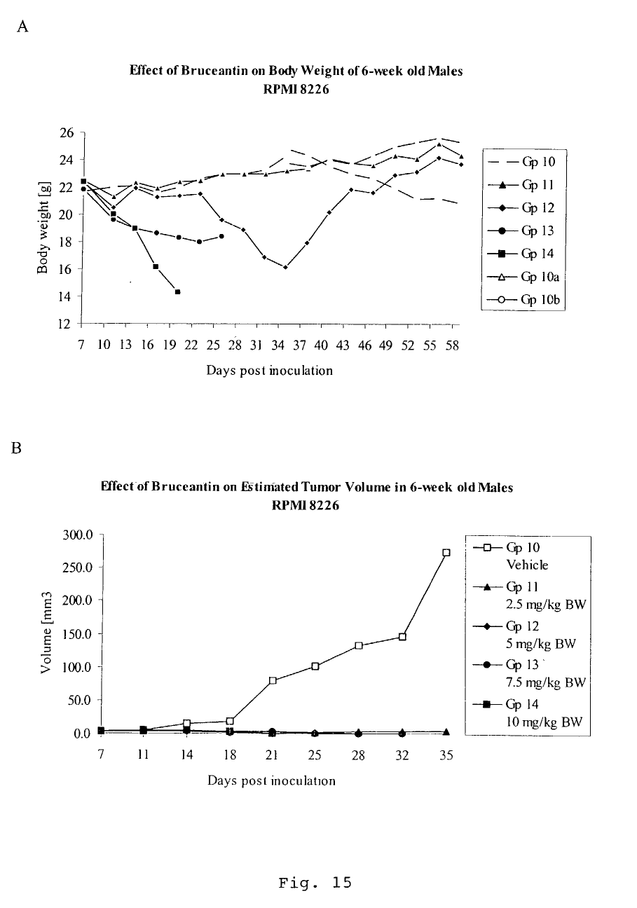

FIGS. 1A and B show the effect of bruceantin administration on mouse body weight and tumor growth, respectively, for Groups 1-5, 1a, and 1b. FIG. 11 shows the positive effects of bruceantin administration in the treatment of multiple myeloma. FIG. 12 shows a reduction of tumor volume for treated-mice (Group 1a) versus control mice (Group 1b), in which tumor volume increased.

Effect of Bruceantin on Estimated Tumor Volume in 13-Week-Old Female SCID Mice Bearing Established RPMI 8226 Tumors

TABLE 9
13-week-old female mice inoculated
with RPMI 8226 cells

| Days | | | | | | |
|---|---|---|---|---|---|---|
| post | | | Tumor | | | Tumor |
| inoculation | BW | Survival | volume | BW | Survival | volume |
| | Group 6 (vehicle) | | | Group 7 (1.25 mg/kg BW) | | |
| 9 | 20.0 | 7 | 2.62 | 20.0 | 8 | 2.84 |
| 12 | 20.0 | 7 | NA | 20.0 | 8 | NA |
| 15 | 20.3 | 7 | 2.38 | 20.4 | 8 | 1.88 |
| 18 | 20.2 | 7 | 5.60 | 20.2 | 8 | 3.50 |
| 21 | 20.2 | 7 | 5.03 | 20.3 | 8 | 2.52 |
| 24 | 20.2 | 7 | 27.23 | 20.2 | 8 | 2.71 |
| 27 | 21.0 | 7 | 28.44 | 20.6 | 8 | 5.13 |
| 30 | 20.1 | 7 | NA | 19.7 | 8 | NA |
| 33 | 20.3 | 7 | 72.62 | 20.4 | 8 | 4.27 |
| 36 | 20.4 | 7 | 109.07 | 20.9 | 8 | 8.88 |
| 39 | 20.1 | 7 | 140.23 | 20.0 | 8 | 22.55 |
| 42 | 20.9 | 7 | 124.43 | 21.2 | 8 | 21.62 |
| 45 | 20.0 | 7 | 218.06 | 19.9 | 8 | 25.88 |
| | Group 8 (2.5 mg/kg EW) | | | Group 9 (5 mg/kg BW) | | |
| 9 | 19.8 | 8 | 2.74 | 20.1 | 8 | 2.56 |
| 12 | 19.5 | 8 | NA1.53 | 19.3 | 8 | NA |
| 15 | 19.9 | 8 | 0 | 19.6 | 8 | 0.94 |
| 18 | 20.0 | 8 | 0 | 19.4 | 8 | 0.98 |
| 21 | 20.0 | 8 | 0 | 20.2 | 7 | 0.43 |
| 24 | 19.9 | 8 | 0 | 19.2 | 6 | 0 |
| 27 | 20.1 | 8 | 0 | 18.9 | 6 | 0 |
| 30 | 19.2 | 8 | NA | 17.5 | 6 | NA |
| 33 | 19.2 | 1 | 0 | 18.2 | 5 | 0 |
| 36 | 19.0 | 8 | 0 | 16.5 | 5 | 0 |
| 39 | 18.9 | 8 | 0 | 16.6 | 5 | 0 |
| 42 | 19.6 | 8 | 0 | 17.1 | 2 | 0 |
| 45 | 18.9 | 8 | 0 | 17.5 | 2 | 0 |

Day 0 - Inoculation of RPMI 8226
Day 7 - First bruceantin injection
Day 42 - Last bruceantin injection for Groups 7-9
Day 45 - First bruceantin injection for Group 6a and no treatment for Groups 7-9.

Forty-two days post inoculation, the mice from Groups 7-9 received a final bruceantin treatment, and the mice from Group 6 were split in two groups: Group 6a containing three mice and Group 6b containing four mice (see FIG. 13). Forty-five days post inoculation, mice in Group 6b were admininstered bruceantin (2.5 mg/kg BW) every 3 days, and mice from Group 6a were administered the vehicle.

FIGS. 13A and B show the effect of bruceantin on mouse body weight and tumor growth, respectively, for Groups 6-9. FIG. 13 shows the positive effects of bruceantin in the treatment of multiple myeloma. FIG. 14 shows a reduction of volume for treated (Group 6b) mice versus control mice (Group 6a), in which tumor volume increased.

Effect of Bruceantin on Estimated Tumor Volume in Six-Week-Old Male SCID Mice Bearing established RPMI 8226 Tumors

TABLE 10
6-week-old male inoculated
with RPMI 8226 cells

| Days | | | | | | |
|---|---|---|---|---|---|---|
| post | | | Tumor | | | Tumor |
| inoculation | BW | Survival | volume | BW | Survival | volume |
| | Group 10 (vehicle) | | | Group 11 (2.5 mg/kg BW) | | |
| 7 | 21.7 | 9 | 3.1 | 22.5 | 10 | 3.4 |
| 11 | 22.0 | 9 | 5.7 | 21.3 | 10 | 4.9 |
| 14 | 22.1 | 9 | 14.9 | 22.3 | 10 | 5.6 |
| 17 | 21.6 | 9 | 19.0 | 21.9 | 10 | 3.2 |
| 20 | 22.0 | 9 | 79.4 | 22.4 | 10 | 2.8 |
| 23 | 22.6 | 9 | 101.2 | 22.5 | 10 | 1.4 |
| 26 | 23.0 | 9 | 132.8 | 23.0 | 10 | 2.7 |
| 29 | 23.0 | 9 | NA | 23.0 | 10 | NA |
| 32 | 23.3 | 9 | 146.3 | 23.0 | 10 | 3.0 |
| 35 | 24.3 | 9 | 274.0 | 23.2 | 10 | 2.8 |
| | Group 12 (5 mg/kg BW) | | | Group 13 (7.5 mg/kg BW) | | |
| 7 | 22.3 | 10 | 2.5 | 21.8 | 10 | 2.8 |
| 11 | 20.5 | 10 | 4.4 | 19.6 | 10 | 3.9 |
| 14 | 21.9 | 10 | 3.2 | 19.0 | 9 | 4.9 |
| 17 | 21.3 | 9 | 2.4 | 18.6 | 8 | 2.0 |
| 20 | 21.4 | 9 | 0 | 18.3 | 8 | 2.8 |
| 23 | 21.5 | 9 | 2.0 | 18.0 | 7 | 0 |
| 26 | 19.6 | 7 | 0 | 18.4 | 5 | 0 |

| 29 | 18.9 | 6 | NA | All mice are dead |
|---|---|---|---|---|
| 32 | 16.9 | 5 | 0 | |
| 35 | 16.2 | 5 | 0 | |
| | Group 14 (10 mg/kg BW) | | | |
| 7 | 22.4 | 10 | 3.7 | |
| 11 | 20.0 | 4 | 2.7 | |
| 14 | 19.0 | 2 | 4.9 | |
| 17 | 16.2 | 1 | 2.5 | |
| 20 | 14.3 | 1 | 0 | |

| | |
|---|---|
| 23 | All mice are dead |
| 26 | |
| 29 | |
| 32 | |
| 35 | |

Day 0 - Inoculation of RPMI 8226
Day 10 - First bruceantin injection
Day 32 - Last bruceantin injection for Groups 11-14
Day 35 - First bruceantin injection for Group 10a and no treatment for groups 11-14
Day 40 - Change dose to 5 mg/kg BW for Group 10b.

Thirty-two days post inoculation, the mice from groups 11-14 received their last bruceantin treatment and the mice from Group 10 were split in two groups: Group 10a containing five mice and Group 10b containing five mice (see FIG. 15). Thirty-five days post inoculation, mice in Group 10b were administered bruceantin (2.5 mg/kg BW) every 3 days, and mice in Group 10a were administered the vehicle. Forty-six days post inoculation, the dose administered to mice from Group 10b was increased to 5 mg/kg BW.

FIGS. 15A and B show the effect of bruce-antin on body weight and tumor growth, respectively, for Groups 10-14. FIG. 15 shows the positive effects of bruceantin in the treatment of multiple myeloma. FIG. 16 shows a reduction of tumor volume for treated mice (Group 10b) versus control mice (Group 10a), in which tumor volume increased.

Preferred agents have an antiproliferative inhibition concentration (IC₅₀ value) of about 1 μM or less, preferably about 0.5 μM or less, with respect to promyelocytic leukemia cells. To achieve the full advantage of the present invention, the chemopreventative or chemotherapeutic agent has an antiproliferative inhibition concentration IC₅₀ of about 0.25 μM or less. Alternatively, preferred agents exhibit a cytotoxicity concentration (IC₅₀ value) of about 0.1 μM or greater, and more preferably of about 0.2 μM or greater. To achieve the full advantage of the present invention, the chemopreventative agent has a cytotoxicity value (IC₅₀) of about 0.3 μM or greater.

In more preferred embodiments, a chemotherapeutic agent of the present invention has a Selectivity Index of 1 or greater, preferably about 1.5 or greater, more preferably about 2 or greater, and most preferably about 3 or greater.

For the purposes of the description herein, the term “treatment” includes preventing, lowering, stopping, or reversing the progression of severity of the condition or symptoms being treated. As such, the term “treatment” includes both medical therapeutic and/or prophylactic administration, as appropriate.

The above tests and data show that brusatol, glaucarubolone, and derivatives thereof can be administered to mammals in methods of treating various cancers. Brusatol, glaucarubolone, and derivatives thereof, as active agents, can be formulated in suitable excipients for oral administration, or for parenteral administration. Such excipients are well known in the art. The active agents typically are present in such a composition in an amount of about 0.1% to about 75% by weight, either alone or in combination.

Pharmaceutical compositions containing an active agent of the present invention are suitable for administration to humans or other mammals. Typically, the pharmaceutical compositions are sterile, and contain no toxic, carcinogenic; or mutagenic compound which would cause an adverse reaction when administered.

Administration of an active agent can be performed before, during, or after exposure to a carcinogen or procarcinogen.

The method of the invention can be accomplished using an active agent as described above or as a physiologically acceptable salt or solvate thereof. The compound, salt, or solvate can be administered as the neat compound, or as a pharmaceutical composition containing either entity.

The active agents can be administered by any suitable route, for example by oral, buccal, inhalation, sublingual, rectal, vaginal, transurethral, nasal, percutaneous, i.e., transdermal, or parenteral (including intravenous, intramuscular, subcutaneous, and intracoronary) administration. Parenteral administration can be accomplished using a needle and syringe, or using a high pressure technique, like POWDERJECT™.

The compounds and pharmaceutical compositions thereof include those wherein the active ingredient is administered in an effective amount to achieve its intended purpose. More specifically, a “therapeutically effective amount” means an amount effective to prevent development of, or to alleviate the existing symptoms of, the subject being treated. Determination of the effective amounts is well within the capability of those skilled in the art, especially in light of the detailed disclosure provided herein.

A “therapeutically effective dose” refers to that amount of the compound that results in achieving the desired effect. Toxicity and therapeutic efficacy of such compounds can be determined by standard pharmaceutical procedures in cell cultures or experimental animals, e.g., for determining the LD₅₀ (the dose lethal to 50% of the population) and the EDs₅₀ (the dose therapeutically effective in 50% of the population). The dose ratio between toxic and therapeutic effects is the therapeutic index, which is expressed as the ratio between LD₅₀ and ED₅₀. Compounds which exhibit high therapeutic indices are preferred. The data obtained from such data can be used in formulating a range of dosage for use in humans. The dosage of such compounds preferably lies within a range of circulating concentrations that include the ED₅₀ with little or no toxicity. The dosage can vary within this range depending upon the dosage form employed, and the route of administration utilized.

The exact formulation, route of administration, and dosage is determined by an individual physician in view of the patient's condition. Dosage amount and interval can be adjusted individually to provide levels of the active agent that are sufficient to maintain therapeutic or prophylactic effects.

The amount of pharmaceutical composition administered is dependent on the subject being treated, on the subject's weight, the severity of the affliction, the manner of administration, and the judgment of the prescribing physician.

Specifically, for administration to a human in the curative or prophylactic treatment of a cancer, oral dosages of an active agent generally are about 0.1 to about 1000 mg daily for an average adult patient (70 kg). Thus, for a typical adult patient, individual tablets or capsules contain 0.2 to 500 mg of an active agent, in a suitable pharmaceutically acceptable vehicle or carrier, for administration in single or multiple doses, once or several times per day. Dosages for intravenous, buccal, or sublingual administration typically are 0.1 to 500 mg per single dose as required. In practice, the physician determines the actual dosing regimen which is most suitable for an individual patient, and the dosage varies with the age, weight, and response of the particular patient. The above dosages are exemplary of the average case, but there can be individual instances in which higher or lower-dosages are merited, and such are within the scope of this invention.

An active agent of the present invention can be administered alone, but generally is administered in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the active agents into preparations which can be used pharmaceutically.

These pharmaceutical compositions can be manufactured in a conventional manner, e.g., by conventional mixing, dissolving, granulating, dragee-making, emulsifying, encapsulating, entrapping, or lyophilizing processes. Proper formulation is dependent upon the route of administration chosen. When a therapeutically effective amount of an active agent of the present invention is administered orally, the composition typically is in the form of a tablet, capsule, powder, solution, or elixir. When administered in tablet form, the composition can additionally contain a solid carrier, such as a gelatin or an adjuvant. The tablet, capsule, and powder contain about 5% to about 95% of an active agent of the present invention, and preferably from about 25% to about 90% compound of the present invention. When administered in liquid form, a liquid carrier such as water, petroleum, or oils of animal or plant origin can be added. The liquid form of the composition can further contain physiological saline solution, dextrose or other saccharide solutions, or glycols. When administered in liquid form, the composition contains about 0.5% to about 90% by weight of an active agent of the present invention, and preferably about 1% to about 50% of an active agent of the present invention.

When a therapeutically effective amount of an active agent of the present invention is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to a compound of the present invention, an isotonic vehicle.

Suitable active agents can be readily combined with pharmaceutically acceptable carriers well-known in the art. Such carriers enable the present compounds to be formulated as tablets, pills, dragees, capsules, liquids, gels, syrups, slurries, suspensions and the like, for oral ingestion by a patient to be treated. Pharmaceutical preparations for oral use can be obtained by adding the active agent with a solid excipient, optionally grinding a resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The active agents can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of the active agents can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

The active agents also can be formulated in rectal compositions, such as suppositories or retention enemas, e.g., containing conventional suppository bases. In addition to the formulations described previously, the compounds also can be formulated as a depot preparation. Such long-acting formulations can be administered by implantation (for example, subcutaneously or intramuscularly) or by intramuscular injection. Thus, for example, the active agents can be formulated with suitable polymeric or hydrophobic materials (for example, as an emulsion in an acceptable oil) or ion exchange resins, or as sparingly soluble derivatives, for example, as a sparingly soluble salt.

In particular, an active agent can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A compound also can be injected parenterally, for example, intravenously, intramuscularly, subcutaneously, or intracoronarily. For parenteral administration, the compound is best used in the form of a sterile aqueous solution which can contain other substances, for example, salts, or monosaccharides, such as mannitol or glucose, to make the solution isotonic with blood.

Modifications and variations of the invention as hereinbefore set forth can be made without departing from the spirit and scope thereof, and, therefore, only such limitations should be imposed as are indicated by the appended claims.

What is claimed is:

1. A method of treating multiple myeloma comprising administering a pharmaceutical composition to a mammal in need thereof, in a sufficient amount to suppress the initiation, promotion, or progression of the multiple myeloma, said composition comprising an active agent selected from the group consisting of brusatol, bruceantin, glaucarubolone, and derivatives thereof.

2. The method of claim 1 wherein the composition is a solid.

3. The method of claim 1 wherein the composition is a liquid.

4. The method of claim 1 wherein the composition is administered orally.

5. The method of claim 1 wherein the composition is administered parenterally.

6. The method of claim 1 wherein the composition is administered prior to exposure of the mammal to a carcinogen or a procarcinogen.

7. The method of claim 1 wherein the composition is administered during or after exposure of the mammal to a carcinogen or a procarcinogen.

8. The method of claim 1 wherein the mammal is a human.

9. The method of claim 8 wherein the composition is administered in a sufficient amount to produce a suppressing effect on the multiple myeloma.

10. The method of claim 1 wherein the composition further comprises an excipient.

11. The method of claim 1 wherein the active agent is present in an amount of about 0.1% to about 75%, by weight of the composition.

12. The method of claim 1 wherein the active agent has an antiproliferative IC₅₀ value versus HL-60 cells of about 2 μM or less.

13. The method of claim 1 wherein the active agent induces apoptosis in RPMI 8226 cells at an IC₅₀ value of about 7 ng/ml or less.

14. The method of claim 1 wherein the active agent has a cytotoxicity IC₅₀ value of 0.1 μM or greater.

15. The method of claim 1 wherein the active agent has a selectivity index of greater than 1.

16. The method of claim 1 wherein the active agent has a selectivity index of greater than 2.

17. The method of claim 1 wherein the active agent has a selectivity index of about 2.5 to about 7.

18. The method of claim 1 wherein the active agent is selected from the group consisting of
Brusatol Series

| | |
|---|---|
| 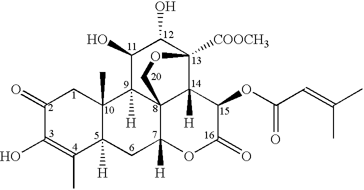 | 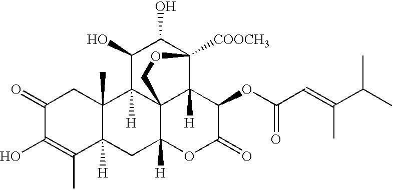 |
| 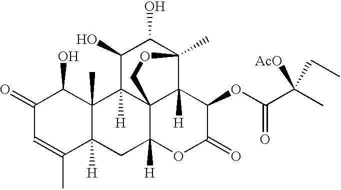 | 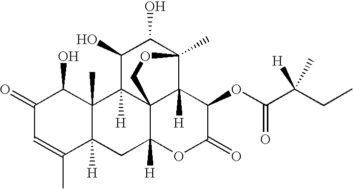 |
| 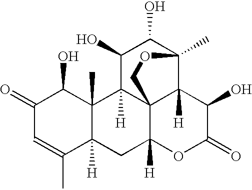 | 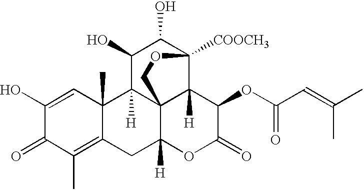 |
| 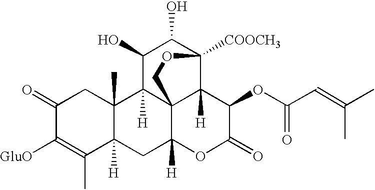 | |

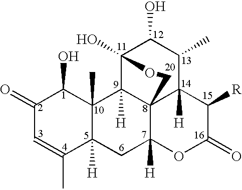

R =

| | | |
|---|---|---|
| 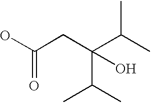 | 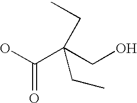 | OH (Claucarubolone) |
| 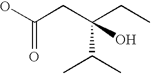 | 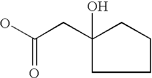 | 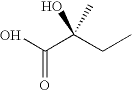 |
| 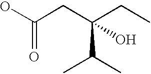 | 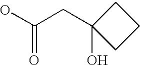 | H (Chaparrinone) |
| 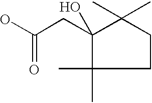 | 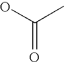 | 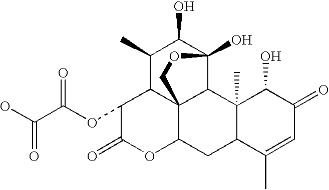 |
| 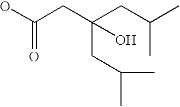 | 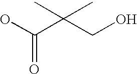 | 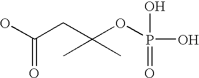 |
| 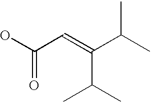 | 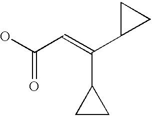 | 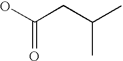 |
| 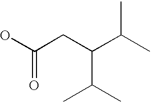 | 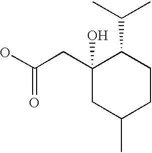 | 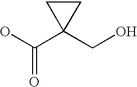 |
| 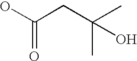 | 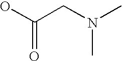 | 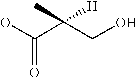 |
| 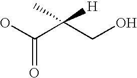 | 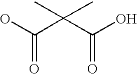 | 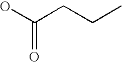 |
| 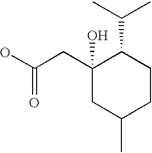 | | |
| | |
|---|---|
| 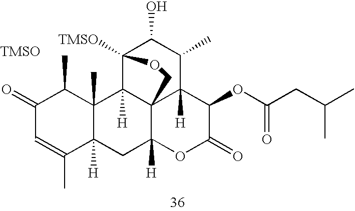 | 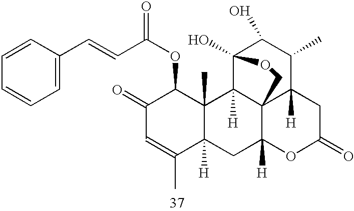 |
| 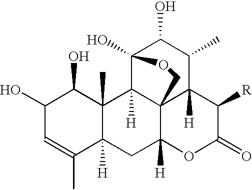 | 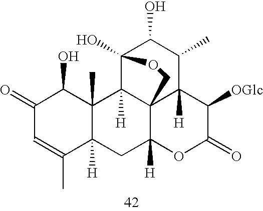 |
| 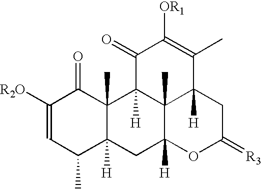 | 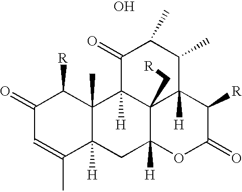 |
| 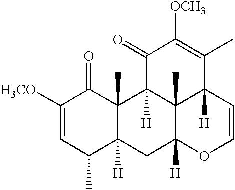 | 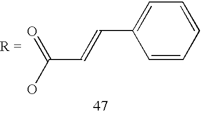 |
| 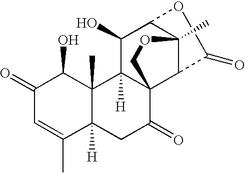 | R = OH |
| R₂ R₃ | R = OGlc |
| CH₃ CH₃ ═O | R = H |
| H H ═O | R = OCOCH₃ |
| CH₃ CH₃ —H, —OH | |

19. The method of claim 1 wherein the active agent is selected from the group consisting of 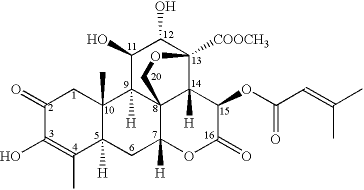 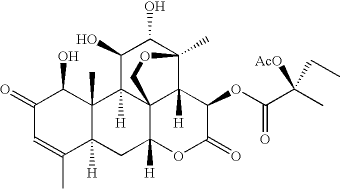 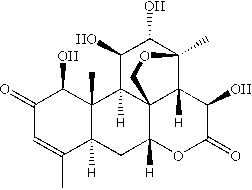 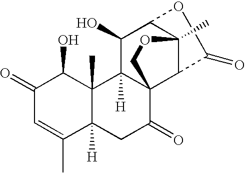 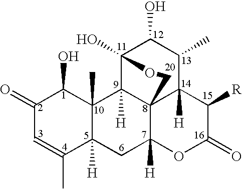
R =

| | |
|---|---|
| 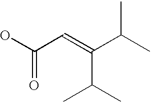 | OH (Glaucarubonlone) |
| 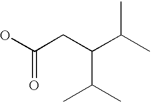 | 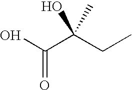 |
| 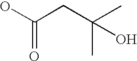 | H (Chaparrinone) |
| 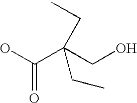 | 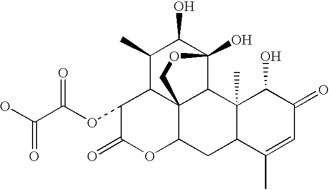 |
| 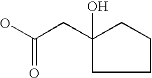 | 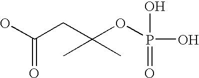 |
| 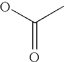 | 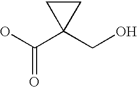 |
| 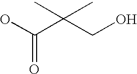 | 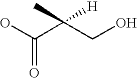 |
| 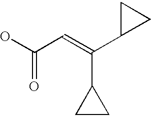 | 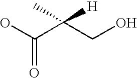 |
| 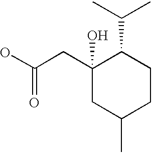 | 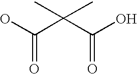 |

20. The method of claim 1 wherein the active agent is selected from the group consisting of 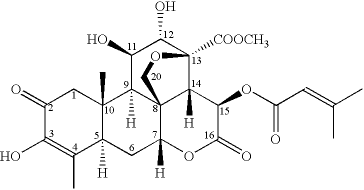 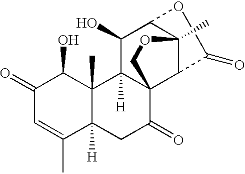 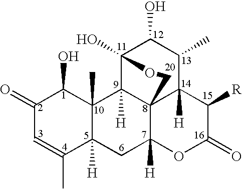
R =

| | |
|---|---|
| 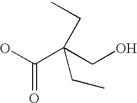 | H (Chaparrinone) |
| 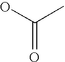 | 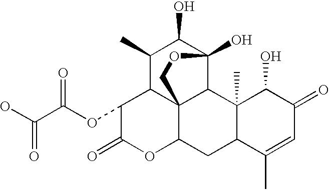 |
| 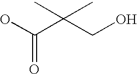 | 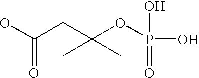 |
| 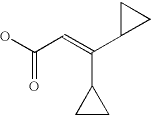 | 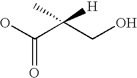 |
| OH (Glaucarubolone) | 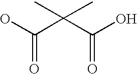 |

21. A method of treating multiple myeloma comprising administering a therapeutically effective amount of an active ingredient to an individual in need thereof, to suppress the initiation, promotion, or progression of the multiple myeloma, said active agent capable of modulating c-Myc or related oncogenes or transcriptase factors.

22. A method of treating a multiple myeloma comprising administering a therapeutically effective amount of an active ingredient to an individual in need thereof, to stimulate differentiation of cancer cells thereby suppressing the initiation, promotion, or progression of the cancer, said active agent capable of modulating c—Myc or related oncogenes or transcription factors.

23. The method of claim 22 wherein a ratio of cytotoxicity IC₅₀ value of the active ingredient to a differentiation EC₅₀ of the active ingredient is greater than 1.0.

24. The method of claim 22 wherein a ratio of a cytotoxicity IC₅₀ value of the active ingredient to a differentiation EC₅₀ of the active ingredient is 2.0 or greater.

25. A method of treating a multiple myeloma comprising administering a therapeutically effective amount of an active quassinoid derivative to an individual in need thereof, to stimulate differentiation of multiple myeloma cells thereby suppressing the initiation, promotion, or progression of the cancer, the said active quassinoid derivative capable of modulating c-Myc or related oncogenes or transcription factors.

26. The method of claim 25 wherein the active quassinoid derivative is selected from the group consisting of brusatol, bruceantin, glaucarubalone, and derivatives thereof.

27. The method of claim 25 wherein the active quassinoid derivative is brusatol or bruceantin.

28. The method of claim 25 wherein the active quassinoid derivative is bruceantin.

29. The method of claim 25 wherein the quassinoid derivative is administered in a sufficient amount to produce a suppressing effect on the multiple myeloma.
